(19) United States
(12) Patent Application Publication
(10) Pub. No.: US 2024/0271187 A1
(43) Pub. Date: Aug. 15, 2024

(54) HYBRIDISATION-BASED SENSOR SYSTEMS AND PROBES

(71) Applicant: Sherlock Biosciences, Inc., Watertown, MA (US)

(72) Inventors: William Jeremy Blake, Winchester, MA (US); Paul David Carlson, Brighton, MA (US); Elizabeth Anne Phillips, Brighton, MA (US); Michael Menglong Liu, Brighton, MA (US); Carl Wayne Brown, III, Brighton, MA (US); Mary Katherine Wilson, Burlington, MA (US); Brendan John Manning, Brighton, MA (US)

(21) Appl. No.: 18/022,098

(22) PCT Filed: Aug. 18, 2021
(86) PCT No.: PCT/US21/46438

§ 371 Date: Feb. 17, 2023

Related U.S. Application Data
(60) Provisional application No. 63/144,887, filed on Feb. 2, 2021.
(60) Provisional application No. 63/067,316, filed on Aug. 18, 2020.

(51) Int. Cl. C12Q 1/6825; C12Q 1/6897

(52) U.S. Cl. CPC C12Q 1/6825; C12Q 1/6897

(57) ABSTRACT

The present disclosure provides compositions and methods that permit detection of nucleic acids in samples (e.g., biological and/or environmental samples). The present disclosure describes “split reporter” sensor system technologies.

### CROSS REFERENCE TO RELATED APPLICATIONS

This application claims priority to each of U.S. Provisional Patent Application Nos. 63/067,316 filed Aug. 18, 2020; 63/144,887 filed Feb. 2, 2021 the entire contents of each of which are hereby incorporated by reference.

### BACKGROUND

Detection of nucleic acids in samples (e.g., biological and/or environmental samples) is increasingly important in a variety of diagnostic, therapeutic, social, and other contexts.

### SUMMARY

The present invention provides certain technologies that permit detection of nucleic acids in samples (e.g., biological and/or environmental samples).

In some embodiments, the present disclosure provides “split reporter” embodiments of sensor system technologies; in some embodiments, use of “split reporter” strategies may improve sensitivity and/or specificity of sensor technologies.

Among other things, the present invention provides improvements to one or more aspects of INSPECTR technologies, e.g., as depicted in one or more of FIGS. 20-25. Certain particular embodiments of provided such technologies are depicted in FIGS. 26-28. As can be seen, provided technologies achieve sensitive detection of target nucleic acid (e.g., nucleic acid of an infectious agent, such as an virus; as exemplified SARS-COV-2)(FIG. 26). Moreover, in some embodiments, provided technologies can be readily multiplexed to achieve simultaneous detection of multiple target nucleic acids (e.g., multiple targets of the same infectious agent, or targets of different infectious agents, as exemplified, SARS-COV-2 and H1N1 Influenza A) (FIGS. 27-28).

Provided technologies can be implemented in a variety of formats, including for example, at central labs, at point of case, and/or at home or in the field. See, for example, FIG. 29.

In some embodiments, the present disclosure provides a nucleic acid sensor system comprising at least a first nucleic acid sensor part and a second nucleic acid sensor part wherein:
(i) the first nucleic acid sensor part comprises:
(a) a sequence that is, encodes, or templates coding of, at least one first reporting element component (e.g., E1); and
(b) a first target hybridization element: and
(ii) the second sensor part comprises:
(a) a sequence that is, encodes, or templates coding of, at least one second reporting element component (e.g., E2); and
(b) a second target hybridization element, wherein: the first and second target hybridization elements are related to one another in that, when the system is contacted with a sample comprising a target nucleic acid, hybridization of the target nucleic acid with both of the first and second target hybridization elements juxtaposes the first and second nucleic acid sensor parts with one another so that the juxtaposed parts are susceptible to linkage by one or more of:
(i) ligation to generate a ligation product:
(ii) templated copying to generate a linked template product.

In some embodiments, the present disclosure provides, a nucleic acid sensor system comprising a plurality of a first nucleic acid sensor parts and a plurality of a second nucleic acid sensor parts wherein:
(i) each of the plurality of the first nucleic acid sensor parts comprises:
(a) a sequence that is, encodes, or templates coding of, at least one reporting element component: and
(b) a target hybridization element; and
   (ii) each of the plurality of the second nucleic acid sensor parts comprises:
(a) a sequence that is, encodes, or templates coding of, at least one reporting element component: and
(b) a target hybridization element.

In some embodiments, the present disclosure provides a method comprising the steps of:
(i) providing a sample comprising the target nucleic acid:
(ii) contacting the sample comprising the target nucleic acid with the nucleic acid sensor system of any of the preceding claims:
(iii) contacting the reaction product of step (ii) with conditions for linking the juxtaposed nucleic acid sensor parts under conditions favorable to the hybridization of the target nucleic acid to the target hybridization elements of the sensor system:
(iv) contacting the reaction product produced in step (iii) with a cell-free expression system, a strand displacing DNA Polymerase, and a primer, under conditions favorable to the production of a reporter.

### BRIEF DESCRIPTION OF THE DRAWING

FIG. 1 demonstrates split reporter technologies as described herein.

FIG. 2 Internal Splint-Pairing Expression Cassette Translation Reaction (INSPECTR) coupled to a dual-epitope peptide reporter In the INSPECTR reaction, de novo-designed DNA sensors ligate to a target nucleic acid (NA), generating a double-stranded DNA expression cassette. Transcription (TX) and translation (TL) generate a peptide output comprised of two epitope domains (E1 and E2). These linked epitopes can then be detected by (for example) dual-antibody ELISA. In this design scheme, E1 and E2 are encoded on different sensor strands: cryptic transcription and translation of unligated sensors generates side-products containing unlinked epitopes which are unable to bind both antibodies to generate a positive signal.

FIG. 3 demonstrates An RNA-based reporter leveraging the split epitope design concept. Ligated expression cassette expresses linked RNA binding domains (B1 and B2) that links surface-immobilized capture oligos and reporter oligos for positive signal detection. To block binding of B1 and B2 DNA domains to capture and reporter oligos, respectively, a cis-repressed hairpin structure is employed for both sensor strands.

FIG. 4 demonstrates INSPECTR detection coupled to Enzyme-Linked Immunosorbent assay (ELISA) readout. Varying concentrations of target RNA were detected by INSPECTR, followed by expression in a cell-free system and detection of linked E1-E2 antibodies by a sandwich ELISA assay (green). Purified dsDNA expressing E1-E2 epitopes was included as an expression control (red).

FIG. 5 demonstrates INSPECTR detection coupled to Lateral Flow (LF) readout. Sensors designed to detect “CT” target RNA successfully encode an E1-E2 peptide that can be visualized by a sandwich LF assay. Incubation of sensors with no target (grey) or a non-target RNA sequence (“FLU”, blue) did not generate visible signal.

FIG. 6 demonstrates Expression of linked E1-E2 epitopes is necessary for detection. Double-stranded DNA encoding linked E1-E2, E1 only, E2 only, or no tag were expressed for 2 h followed by detection by ELISA. Only when linked epitopes are expressed is significant signal-above-background observed.

FIG. 7 demonstrates Concatenation of epitope tags for improved antibody binding efficiency Epitope tags (E1 and/or E2) can be expressed in tandem (2 or more tags in series) to improve detection sensitivity.

FIG. 8 demonstrates Concatenated epitope tags improves output signal. Equimolar concentrations of dsDNA encoding monomeric E1-E2 tags or tandem E1-E1-E2-E2 tags were expressed for 2 h and detected by ELISA. Higher signal is observed when tandem tags are incorporated.

FIG. 9 demonstrates tandem tags improve sensitivity.

FIG. 10 demonstrates concatenated Myc epitope tags improved output signal.

FIG. 11 demonstrates concatenated FLAG epitope tags improves output signal.

FIG. 12 demonstrates concatenated HA epitope tags improve output signal.

FIG. 13 demonstrates use of methods and compositions described herein with a tethered split protein output.

FIG. 14 demonstrates exemplary split proteins for A and B signal integration.

FIG. 15 demonstrates exemplary peptide cleavage for A and not B signal integration.

FIG. 16 demonstrates multiplexed target detection with a lateral flow readout.

FIG. 17 demonstrates multiplexed detection with a lateral flow readout.

FIG. 18 demonstrates detection of multiple subdomains within a larger target nucleic acid strand.

FIG. 19 demonstrates recycling epitope domains for multiplexed target

detection.

FIG. 20 demonstrates the INSPECTR principle.

FIG. 21 demonstrates cell-free expression systems.

FIG. 22 demonstrates INSPECTR Process and Advantages.

FIG. 23 demonstrates bacterial detection with luminescent readout.

FIG. 24 demonstrates INSPECTR single base specificity.

FIG. 25 demonstrates compositions and methods described herein are programmable for Lateral Flow Detection.

FIG. 26 demonstrates SARS-COV-2 RNA Detection using methods and compositions described herein.

FIG. 27 demonstrates compositions and methods described herein are multiplexable.

FIG. 28 demonstrates compositions and methods described herein are

useful for independent detection of respiratory pathogen RNA.

FIG. 29 demonstrates compositions and methods described herein are

useful in varied applications.

FIG. 30 provides an exemplary schematic demonstrating how addition of a gap-filling oligo (GFO) can mitigate the effects of off-target DNA ligation. A. In an exemplary, typical nucleic acid (NA) detection reaction, DNA sensors (e.g., hybA and hybB) bind the NA target, forming a splinted substrate for DNA ligase. Formation of a ligated protein (hybA+hybB) can be used as a positive indicator for the presence of a NA target. In some embodiments, ligase promiscuity can also lead to off-target sensor ligation in the absence of a target NA. This off-target product is indistinguishable from the correct (e.g., on-target) ligation product which can compromise the utility of this approach for NA detection. B. In an exemplary, alternative design configuration, sensor hybridization regions are truncated (hybA_{T} and hybB_{T}), resulting in a gap between the two sensor ends when the NA target is splinted. An additional DNA strand (e.g., a gap-filling oligo, GFO) can bridge a can between said ends. Two separate ligation events (on either side of a GFO) must occur to generate a completed probe reporter. A requirement for multiple ligation events, in some embodiments, in a specified order, reduces the probability of generating the correct probe reporter in the absence of target NA.

FIG. 31 provides an exemplary schematic demonstrating use of multiple GFOs to bridge longer sequences gaps. In some embodiments, a hybA_{T} and hybB_{T} gap sequence can be bridged by multiple GFOs, which are bound and ligated in tandem (n≥2). Additional GFOs can result in higher stringency by requiring many ligation events, in some embodiments, in a specific order, to generate a correct probe reporter.

FIG. 32 provides an exemplary schematic demonstrating GFO-filled product discrimination from off-target products via qPCR selection primers. In some embodiments, sequence differences between on-target and off-target ligation products generated via GFO-based sensors may be resolved by qPCR. qPCR amplification primers or probes are designed to target sensor-GFO junction regions: as a result, only ligation products generated in the presence of target NA are detected.

FIG. 33 provides an exemplary schematic demonstrating selective amplification of GFO-filled product by isothermal amplification. In some embodiments, sequence differences between on-target and off-target ligation products generated via GFO-based sensors may be resolved by isothermal amplification. Amplification primers or probes are designed to target sensor-GFO junction regions: as a result, only ligation products generated in the presence of target NA are amplified.

FIG. 34 provides an exemplary schematic demonstrating GFO design enabled translational proofreading via generation of a frameshifted reporter peptide in the absence of GFO incorporation. A. In an exemplary, typical, Internal Splint-Pairing Expression Cassette Translation Reaction (INSPECTR), DNA sensors encoding a reporter peptide are ligated in the presence of a NA target, generating a dsDNA expression cassette. Sensor strands (encoding domains E1 and E2, respectively) may be ligated either in trans (forming a linear expression cassette) or in cis (forming a circular expression cassette). Non-templated ligation of a sensor generates the same expression cassette as the on-target product which can result in undesirable transcription (TX) and translation (TL) of functional off-target product. B. Applying a GFO design to INSPECTR can enable translational proofreading of mis-ligated products. In the presence of a target NA, a GFO can be ligated between E1 and E2 sensors. Importantly, the length of this GFO may be any non-multiple of 3 nucleotides. As a result, non-templated ligation generates a truncated expression cassette and the reporter peptide is frameshifted after the E1 domain, generating a non-functional reporter. In some embodiments, reporter detection is carried out by sandwiched antibody binding domains E1 and E2, although GFO proofreading may be applied to any protein or peptide-based reporter system.

FIG. 35 provides an exemplary schematic of GFO designs which may incorporate additional sequence or sequence domain(s). A. GFOs, for example, can form a continuous duplex with a subdomain of a target NA. B. GFOs, for example, may include additional non-binding sequences or sequence domain(s) within a GFO, forming a three-way junction (3WJ) or other non-duplex structure with a target NA. This enables additional design flexibility by selectively incorporating functional (e.g. transcriptional promoters, ribosome binding sites) or structural (e.g., DNA hairpins, primer-binding domains) sequence elements only upon incorporation of a GFO.

FIG. 36 demonstrates exemplary implementation of a GFO to minimize background signal from a dual-epitope peptide (DEP) reporter. In the absence of target NA, a frameshifted reporter peptide was generated and no visible test line from a lateral flow readout was detected. In the presence of target NA, a correct (e.g., non-frameshifted) reporter was generated and produced a robust visible test line from a lateral flow readout.

FIG. 37 demonstrates GFO proofreading can enable sensitive qPCR-based detection of a target RNA. A. Exemplary experimental design to characterize the specificity of GFO designs linked to a qPCR readout. Two ends of a ssDNA probe (domains a and b) bind to a target RNA comprising a complementary sequence b, c, and a, wherein a GFO with a sequence complementary to sequence c fills a gap to generate a ligateable substrate. B. An exemplary DNA ligation reaction was carried out in the presence of on-target RNA sequence (CP=correct product) and/or a fragmented RNA product (complementary b, a sequence) to selectively generate mis-ligated product (OTP=off-target product). C. Ligation product was detected by qPCR primers designed to bind across GFP-probe ligation junctions. Even in the presence of high levels of mis-ligated product, only conditions containing the correct RNA target sequence were detected by qPCR. As negative controls, a No Phosphorylation (No Phos) condition, in which sensor could not be ligated, and a No RNA Target (NTC) condition were utilized.

FIG. 38 demonstrates exemplary, alternative GFO configurations can maintain their ability to selectively ligate only in the presence of target NA. Exemplary, alternative GFO configurations were ligated in the presence of an RNA target and detected via qPCR readout using a circular probe backbone. Gap length (10 nucleotides, 34 nucleotides), number of GFOs (1-4), and addition of non-binding sequence within the GFO (T7 RNA polymerase promoter) were tested with increasing concentrations of RNA target. All conditions demonstrated undetectable signal in the absence of target. A concentration-dependent detectable signal was observed in the presence of increasing concentrations of target NA.

FIG. 39A-B demonstrates exemplary detection of multiple sub-regions within a larger nucleic acid strand. A. A plurality of first and second nucleic acid sensor parts can be utilized targeting sub-regions of the same target nucleic acid to increase sensitivity. B. Four sets of first and second nucleic acid sensor parts targeting different regions of the same target nucleic acid show increased signal compared to a detection assay utilizing only a single set of first and second nucleic acid sensor parts selected from the set of four.

FIG. 40A-D demonstrates exemplary use of first and second nucleic acid sensor parts in a circular configuration. A. A circular configuration can enable improved sensitivity via multiple-turnover amplification by a DNA polymerase. B. Amplification of an exemplary first and second nucleic acid sensor parts in a circular configuration shows accumulation of a large, concatenated amplicon in as little as 15 minutes at 22° C. C. Amplified target nucleic acid was measured over time using quantitative Polymerase Chain Reaction (qPCR). Amplification increases over the course of a 20 hour reaction. D. qPCR-based assessment showed that increased temperature increases amplification rate, consistent with the optimal temperature (30° C.) for the DNA polymerase utilized.

FIG. 41 demonstrates circular configuration use of first and second nucleic acid sensor parts can also increase sensitivity with a lateral-flow based read out. Use of a circular configuration showed about 105-fold improved sensitivity compared to a linear configuration when detected via an expressed peptide reporter.

FIG. 42A-C demonstrates use of gap-filling oligos (GFOs). A. GFOs can introduce a frameshift to provide proofreading. Circularized first and second nucleic acid sensor parts were generated to correspond to a correctly generated-ligation product (FIG. 42A, top) and an incorrectly generated-ligation product (FIG. 42B, bottom). Two GFOs of different lengths were tested, each designed to introduce a frameshift in a reporter peptide upon mis-ligation. B. Primers that could hybridize to both the correctly generated and incorrectly generated-linked products efficiently amplified both the correctly generated and incorrectly generated-linked products. C. Designed proofreading prevents detection of mis-linked product.

FIG. 43A-L demonstrates moving different sequence elements onto GFOs can reduce signal from mis-ligation events. A. When a frameshift-inducing GFO is used in combination with a highly sensitive circularized first and second nucleic acid sensor parts, some background level signal (e.g., signal similar to that of a no target nucleic acid comprising sample). B-F. Example configurations of the distribution of various sequence elements across probe domains, which in most cases reduces process background beyond use of a GFO alone. G-L. Signal output of A-F, respectively. Each column shows the signal output for progressively decreasing concentration of target nucleic acid relative to control (no target control).

FIG. 44A-D provides exemplary experimentation demonstration that a plurality of regions that can hybridize to a target nucleic acid can be incorporated into first and second nucleic acid sensor parts. A. Utilizing a GFO-based approach, a circular first and second nucleic acid sensor parts can be divided into two halves that can be linked (e.g., by ligation, templated copying to generate a linked template product) to form a linked template product. B. Such a configuration promotes logic integration (e.g., wherein both of a target nucleic acid A and B must be present for a linked-product of the circular first and second nucleic acid sensor parts to be generated). C. Target nucleic acid sequences A and B are the same sequence. D. Target nucleic acid sequences A and B are different sequences.

FIG. 45 demonstrates that exonucleases can also be utilized to degrade non-linked first and second nucleic acid sensor parts. When linked-circular first and second nucleic acid sensor parts are hybridized to a target nucleic acid, excess non-linked circular first and second nucleic acid sensor parts remain that can be degraded by exonuclease enzymes while the linked-circular first and second nucleic acid sensor parts remain unaffected.

FIG. 46 demonstrates that exemplary design of structure amplification primers can improve polymerization efficiency.

FIG. 47 demonstrates exemplary design of amplification primers comprising sequences that can hybridize to a plurality of target nucleic acids.

FIG. 48A-B demonstrates exemplary validation of a His tag as a compatible element of the split epitope reporter (E1 or E2, as in FIG. 3), either as the capture (A) or detection (B) antibody of a sandwich-based lateral flow (LF) readout. Anti-His monoclonal antibodies functionalized on nitrocellulose membrane capture (A) peptide expressed from InM of a dsDNA expression cassette template with a His tag. Similarly, anti-His monoclonal antibodies functionalized on colloidal gold yield detection of (B) peptide expressed from InM of expression cassette template. When used as the detection antibody, the His tag can be placed on either the N-terminus or C-terminus of the peptide.

FIG. 49 demonstrates an exemplary lateral flow-based readout is highly specific for multiplexed dual-epitope peptide readouts. A lateral flow strip was developed to capture two representative dual-epitope peptide reporters, differing only in one of their two epitopes (V5-StrepII and FLAG-StrepII). Test lines were generated using anti-V5 and anti-FLAG antibodies, respectively, and a single StrepII detection antibody was employed for both peptides. Purified peptides were then detected by these strips, either individually or pooled. Appearance of test lines were found to be highly specific for their corresponding test lines.

FIG. 50 provides exemplary demonstration that dual-epitope peptide reporters are compatible with a wide array of target regions. Double-stranded DNA cassettes encoding FLAG-StrepII dual-epitope peptides with target regions against 187 unique subdomains of the SARS-COV-2 genome were expressed in a cell-free reaction and then detected by a lateral flow test strip. Every sensor variant was detectable at a concentration of 10 pM DNA in the cell-free reaction, demonstrating that this reporter configuration is robust against sequence changes.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological-setting), so that, for example, one or more features of specificity is determined. In some embodiments, affinity is assessed relative to a reference (e.g., that has a known affinity above a particular threshold [a “positive control” reference] or that has a known affinity below a particular threshold [ a “negative control” reference]. In some embodiments, affinity may be assessed relative to a contemporaneous reference: in some embodiments, affinity may be assessed relative to a historical reference. Typically, when affinity is assessed relative to a reference, it is assessed under comparable conditions.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond: two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal: in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies: bi- or multi- specific antibodies (e.g., Zybodies R, etc): antibody fragments such as Fab fragments, Fab′ fragments, F(ab′)2 fragments, Fd′ fragments, Fd fragments, and isolated CDRs or sets thereof: single chain Fvs: polypeptide-Fc fusions: single domain antibodies (e.g., shark single domain antibodies such as IgNAR or fragments thereof): cameloid antibodies: masked antibodies (e.g., Probodies R:): Small Modular ImmunoPharmaceuticals (“SMIPs™”): single chain or Tandem diabodies (TandAbR:): VHHs: Anticalins®: Nanobodies ®; minibodies: BiTE®s: ankyrin repeat proteins or DARPINS®: Avimers®; DARTs: TCR-like antibodies:, Adnectins®: Affilins®: Trans-bodies®: Affibodies®: TrimerX®: MicroProteins: Fynomers®, Centyrins®: and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another: in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptaviding and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers: in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Characteristic sequence: A “characteristic sequence” is a sequence that is found in all members of a family of polypeptides or nucleic acids, and therefore can be used by those of ordinary skill in the art to define members of the family.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide: those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Detectable entity: The term “detectable entity.” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²p, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic”amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | Z | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position: when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna. CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature: b) it is substantially free of other polypeptides or nucleic acids of the same species from the species that produces it in nature: c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature: and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest: in some embodiments, control elements act in trans to or otherwise at a from the functional element of interest.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner: in some embodiments, specific binding is assessed by detecting or determining degree of dissociation of a binding agent-partner complex: in some embodiments, specific binding is assessed by detecting or determining ability of the binding agent to compete an alternative interaction between its partner and another entity. In some embodiments, specific binding is assessed by performing such detections or determinations across a range of concentrations.

### Detailed Description of Certain Embodiments

### Sensor Systems

In various embodiments, the present disclosure provides systems that utilize nucleic acid sensor technologies that initially (and target-sequence dependent) hybridize to target nucleic acids of interest, and uses those systems to generate a detectable output.

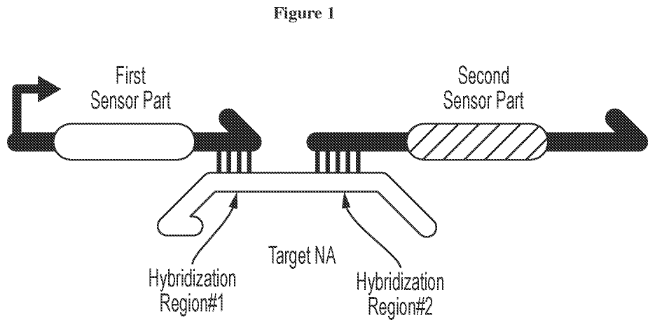

FIG. 1 depicts an exemplary embodiment of the nucleic acid sensor system in accordance with the present disclosure. As indicated in FIG. 1, a nucleic acid sample that may include at least one nucleic acid of interest (i.e., whose nucleotide sequence is or includes a target site of interest) is contacted with a set of nucleic acid sensors. A nucleic acid sensor set comprises at least a first nucleic acid sensor part and a second nucleic acid sensor part wherein (i) a first nucleic acid sensor part comprises (a) a sequence that is, encodes, or templates at least one reporting element and (b) a first hybridization element: and (ii) a second nucleic acid sensor part comprises (a) a sequence that is, encodes, or templates at least one reporting element and (b) a first hybridization element. The first and second nucleic acid sensor parts are related to one another in that, when the system is in contact with a sample comprising a target nucleic acid, hybridization of the target nucleic acid with both of the first and second nucleic acid sensor parts juxtaposes the first and second nucleic acid sensor parts with one another so that the juxtaposed parts are susceptible to linkage by one or more of (i) ligation to generate a ligation product and/or (ii) templated coping to generate a linked template product (e.g., they form a “nicked arrangement”).

In some embodiments, one or both nucleic acid sensor parts may comprise a templating element that directs synthesis of a single, intact strand complementary to the nicked arrangement. For example, where a templating element is or comprises a promoter and/or one or more transcriptional regulatory elements, the system may be or comprise an RNA polymerase: where a templating element is or comprises an origin of replication and/or a binding site for an extendible primer, the system may be or comprise a DNA polymerase (which, in some embodiments, may be a thermostable DNA polymerase, particularly if the juxtaposed strand includes a sequence element corresponding to a second extendible primer and the system includes an appropriate pair of primers to amplify a duplex of the juxtaposed strand and its complement).

In some embodiments, linkage of first and second nucleic acid sensor parts generates a nucleic acid strand (i.e., a linked strand) that includes both of the first and second reporting elements (or their complements), which nucleic acid strand is a reporter in that it, or its complement (e.g., generated by transcription or extension [e.g., primed extension]), or an expression product (e.g., generated by transcription and/or translation) of either, is detectable or otherwise generates or participates in generation of a detectable signal indicative of presence and/or amount of the target nucleic acid in the sample.

In some embodiments, a linked strand may be transcribed and/or translated (e.g., via cell-free components such as a cell free protein synthesis expression system (CFPS)).

In some embodiments, linkage as described herein generates a detectable output. In some embodiments, such detectable output is or is generated by a polypeptide. In some embodiments, a detectable output may be or comprise a catalytic output: in some embodiments, a detectable output may be or comprise a non-catalytic output.

In some embodiments, a catalytic output is or is generated by an enzyme that catalyzes a reaction, e.g., converting one or more substrates to one or more detectable. In some embodiments, a non-catalytic output is or generates a detectable nucleic acid or polypeptide (e.g., that act as an antigen or other specific binding ligand).

Among other things, the present disclosure provides technology formats in which a detectable output is amenable to lateral flow analysis (e.g., is, comprises, or generates a product that is detectable by lateral flow). In some embodiments, the present disclosure provides an insight that coupling linkage mediated detection technologies with lateral flow assessment technologies may particularly facilitate multiplexed analyses (e.g., simultaneous detection of a plurality of products amenable to lateral flow). Furthermore, the present disclosure teaches that such coupling may have particular advantages that permit effective multiplexed analysis of products of different chemical class (e.g., two or more of nucleic acids, metals, polypeptides, small molecules, antibodies or fragments thereof etc.).

Below, certain features and/or attributes of various elements and/or embodiments of the present invention are discussed in more detail. Those of ordinary skill, reading the disclosure, will be aware of certain modifications and/or variations that are within the skill of one of ordinary skill and also are within the spirit and scope of this disclosure. Those of ordinary skill will also appreciate that, given the templating property of nucleic acid strands, and their ability to hybridize with one another, it is conventional in the field to refer to a particular “sequence element” or “site” and relying on the skilled artisan to understand from context when the “forward” or “sense” form is being referenced, and when its complement (the “reverse” or “antisense” form).

### Sensor Parts

### Target Hybridization Elements

As depicted in FIG. 1, nucleic acid sensor sets for use in accordance with the present disclosure are designed so that each includes a sequence element that hybridizes to a target nucleic acid at a position adjacent to that where another nucleic acid sensor of the set, such that when all nucleic acid sensors of a particular set are hybridized to a target nucleic acid (i.e., to a nucleic acid that includes a target site), they can be covalently linked to one another by one or more of (a) ligation to generate a ligation product and/or (b) templated coping to generate a linked template product (e.g., they form a “nicked arrangement”). In some embodiments, a sensor system as provided herein may include one or more bridging oligonucleotides (e.g., which may be referred to as “gap filling” oligonucleotides (“GFO”) that hybridize to the target site between other, e.g., the first and second, nucleic acid sensor parts.

In some embodiments, a set of nucleic acid sensors includes only two (i.e., first and second) nucleic acid sensor parts, each of which includes a target hybridization element and one of which includes a primer element: in some embodiments, the other includes a templating element. In some embodiments, a set of nucleic acid sensors includes one or more bridging oligonucleotides that hybridize to the target site between the first and second nucleic acid sensors.

In some embodiments, a GFO can reduce background or off-target signal. In some embodiments, no detectable output is generated by ligation of a first nucleic acid sensor part and a second nucleic acid sensor part in the absence of a GFO. In some embodiments, an output generated by ligation of a first nucleic acid sensor part and a second nucleic acid sensor part in the absence of a GFO is not a reporting element. In some embodiments, an output generated by ligation of a first nucleic acid sensor part and a GFO or a second nucleic acid sensor part and a GFO is not a reporting element.

In some embodiments, a GFO comprises a primer element. In some embodiments, linkage of a first nucleic acid sensor part, a second nucleic acid sensor part, and a GFO generates a nucleic acid strand (i.e., a linked strand) that includes both of the first and second reporting elements (or their complements), which nucleic acid strand is a reporter in that it, or its complement (e.g., generated by transcription or extension [e.g., primed extension]), or an expression product (e.g., generated by transcription and/or translation) of either, is detectable or otherwise generates or participates in generation of a detectable signal indicative of presence and/or amount of the target nucleic acid in the sample. In some embodiments, a linked strand is amplified (e.g., by a polymerase chain reaction e.g., isothermeral rolling circle amplification). In some embodiments, amplification utilizes at least a primer element in a GFO.

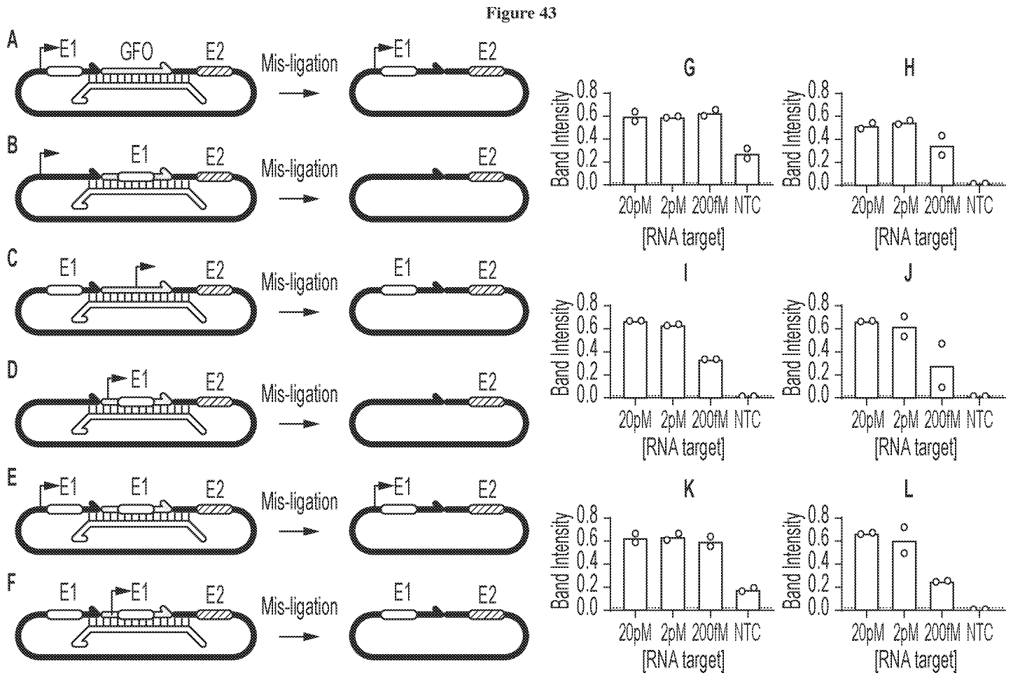

In some embodiments, a GFO comprises one or more nucleic acid sensor parts comprising one or more sequences that is/are, encodes, or templates at least one reporting element (e.g., E1 and/or E2: FIG. 43). In some embodiments, a GFO comprising one or more sequences that is/are, encodes, or templates at least one reporting element

In some embodiments, a first nucleic acid sensor part comprises an E1-encoding sequence. In some embodiments, a second nucleic acid sensor part comprises an E2-encoding sequence. In some embodiments, a first nucleic acid sensor part comprises a plurality of E1-encoding sequences. In some embodiments, a second nucleic acid sensor part comprises a plurality of E2-encoding sequences.

In some embodiments, a first nucleic acid sensor part and a second nucleic acid sensor part are directly linked. In some embodiments, a first nucleic acid sensor part and a second nucleic acid sensor part are indirectly linked (e.g., by a third and fourth target hybridization element and/or GFO). In some embodiments, a first nucleic acid sensor part and a second nucleic acid sensor part being linked (e.g., directly or indirectly) forms a circular probe. In some embodiments, a circular probe comprises one target hybridization element. In some embodiments, a circular probe comprises a plurality of target hybridization elements.

Those skilled in the art, reading the present disclosure, will appreciate that the target site can be any sequence of interest—e.g., whose presence in a particular sample is to be assessed. Those skilled in the art will further be aware (e.g., from other nucleic acid sensor systems described in the literature, including those cited herein) of design considerations relevant to selecting length and/or sequence characteristics (e.g., GC content etc) of individual target hybridization elements in oligonucleotides within a set of nucleic acid sensors.

In some embodiments of the present disclosure, for example, where multiple nucleic acid sensor sets (i.e., designed for detection of different target nucleic acids) are to be used simultaneously (e.g., together in the same reactions), it may be desirable that different nucleic acid sensor sets have different primer elements (e.g., each oligonucleotide set may have its own primer element), but in some embodiments, multiple different nucleic acid sensor sets, or even all nucleic acid sensor sets, may include the same primer element.

### Reporting Element

In many embodiments, sensor systems as described herein include at least first and second sensor parts, each of which includes a reporting element. Operation of provided sensor systems generates a reporter from the reporting elements (i.e., by linking the sensor parts to one another [e.g., by ligation or templating], and, optionally further by copying (e.g., amplifying) and/or expressing (e.g., transcribing and/or translating) the reporting elements).

In some embodiments, a reporter is a nucleic acid (e.g., a nucleic acid strand or duplex).

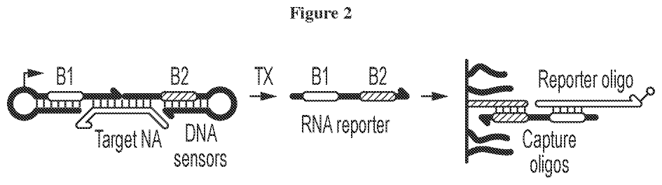

In some embodiments, a reporter nucleic acid comprises at least first and second hybridization elements (see. e.g., FIG. 2), each of which may have a nucleotide sequence that is the same as, or the complement of, one of the reporting elements in a sensor part. Alternatively, a reporter nucleic acid in some embodiments may comprise a single hybridization site, having a nucleotide sequence that is the same as or the complement of, the combination of reporting elements (and, in this case, the reporting elements extend through the hybridization elements) of the sensor parts. As yet another alternatively, in some embodiments, a reporter nucleic acid may comprise a plurality of hybridization sites, one or more of which may have a nucleotide sequence that is the same as, or the complement of, one of the reporting elements, and at least one of which may have a nucleotide sequence that is the same as or the complement of, the combination of reporting elements (and, in this case, the reporting elements extend through the hybridization elements) of the sensor parts.

In some embodiments, a reporter is a nucleic acid that may have enzymatic activity (e.g., that is not present in either of the separate reporting elements present in the sensor parts.

In some embodiments, a reporter is or comprises a polypeptide that includes sequences encoded by each of the reporting elements (or their complements); that is, each reporting element is or comprises an encoding element (i.e., a sequence that encodes, or templates encoding of, a polypeptide or portion thereof).

In some embodiments, such a reporter polypeptide includes at least one epitope or other binding site that specifically interacts with a binding partner (e.g., one epitope or site formed by sequences encoded by each of the reporting elements (or their complements), or at least two epitopes or sites [e.g., optionally encoded by different reporting elements (or their complements)].

In some embodiments, a reporter is or comprises a polypeptide with enzymatic activity.

In some embodiments, a reporter polypeptide is selected from the group consisting of luciferase, beta-lactamase, beta-galactosidase, horseradish peroxidase, alkaline phosphatase, catalase, carbonic anhydrase, green fluorescent protein, red fluorescent protein, cyan fluorescent protein, yellow fluorescent protein, trypsin, a protease, a peptide that complements and activates a truncated reporter protein, a kinase. In other embodiments, the systems include one or more of ligase, a strand-displacing DNA polymerase, dNTPs, RNAse inhibitor, and a cell free expression system. In other embodiments, the systems may also include a reverse transcriptase with a functional RNaseH domain. In other embodiments, the systems may include separate reverse transcriptase and RNaseH activities. In other embodiments, the cell free expression system is whole cell extract. In other embodiments, the strand displacing DNA polymerase is selected from the group consisting of Klenow fragment with exonuclease portion, Klenow fragment without the exonuclease portion, phi29 polymerase, and a modified T7 DNA polymerase.

### Hybridization Elements

In some embodiments, a hybridization element may hybridize to a capture nucleic acid, e.g., that may be associated with a solid phase (see, e.g., FIG. 2).

In some embodiments, a hybridization element may hybridize to a labeled reporting nucleic acid (see. e.g., FIG. 2).

In some embodiments, a hybridization element may hybridize with a CRISPR/Cas gRNA. In some particular embodiments, a hybridization element may hybridize with a CRISPR/Cas gRNA to recruit a Cas protein (e.g., a Cas12 or Cas13 protein) characterized by collateral activity (which may, for example, cleave a reporting nucleic acid to generate a detectable signal).

In some embodiments, a hybridization element may be or comprise a templating element.

### Encoding Elements

(i) Epitopes. In some embodiments, as described herein, an encoding element encodes (or templates encoding of) an epitope or portion thereof, or a binding moiety that specifically interacts with a partner other than an immunoglobulin. In some embodiments, an epitope may be a linear epitope: in some embodiments, an epitope may be a conformational epitope.

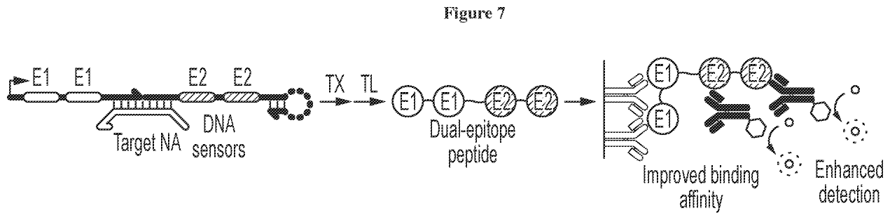

In some embodiments, the reporter is a polypeptide that may include tandem epitopes. In some embodiments, tandem epitopes may improve detectable output of the disclosed system (FIG. 7).

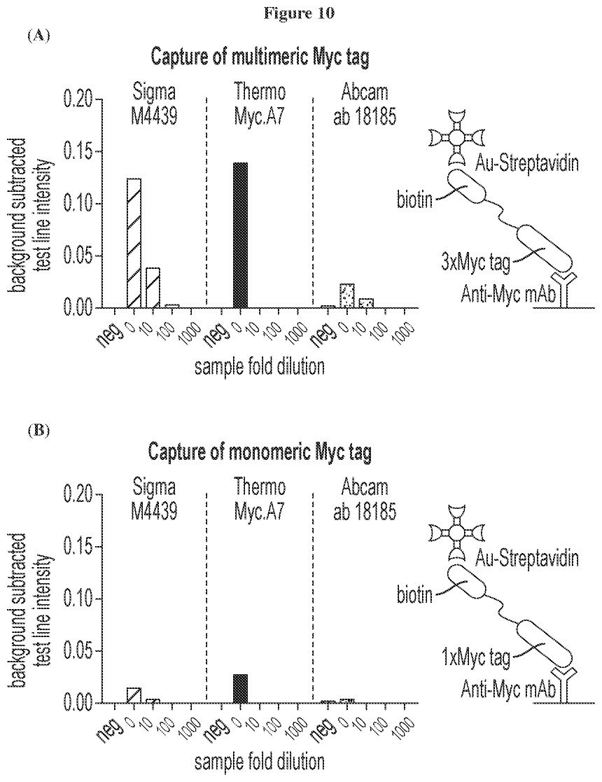

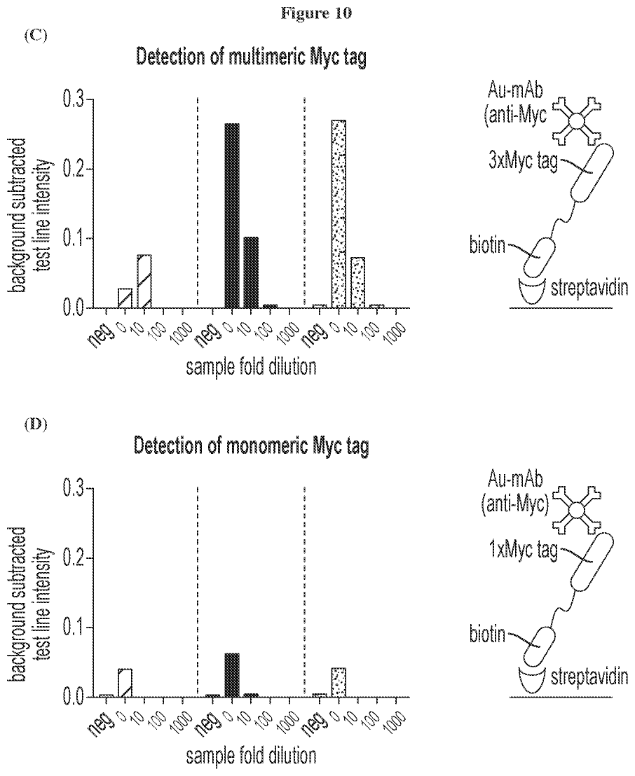

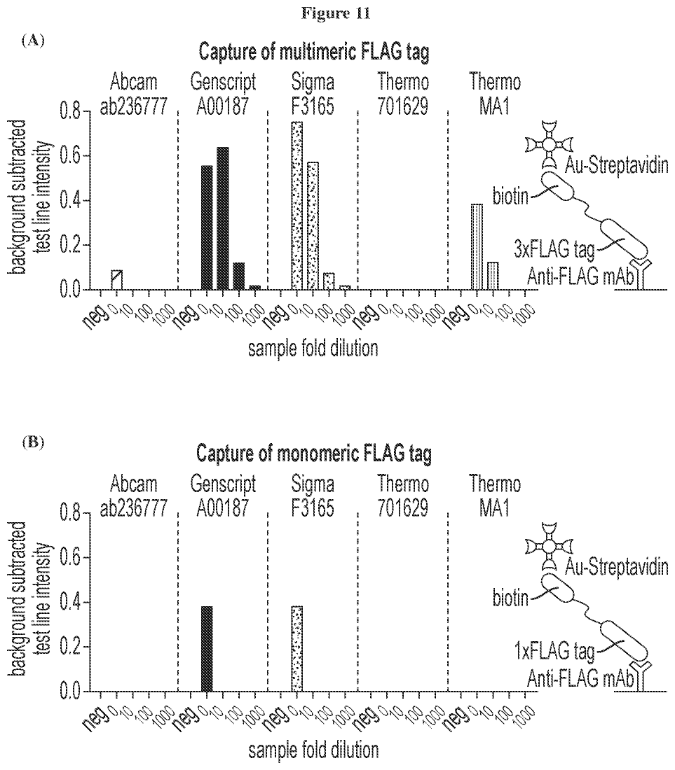

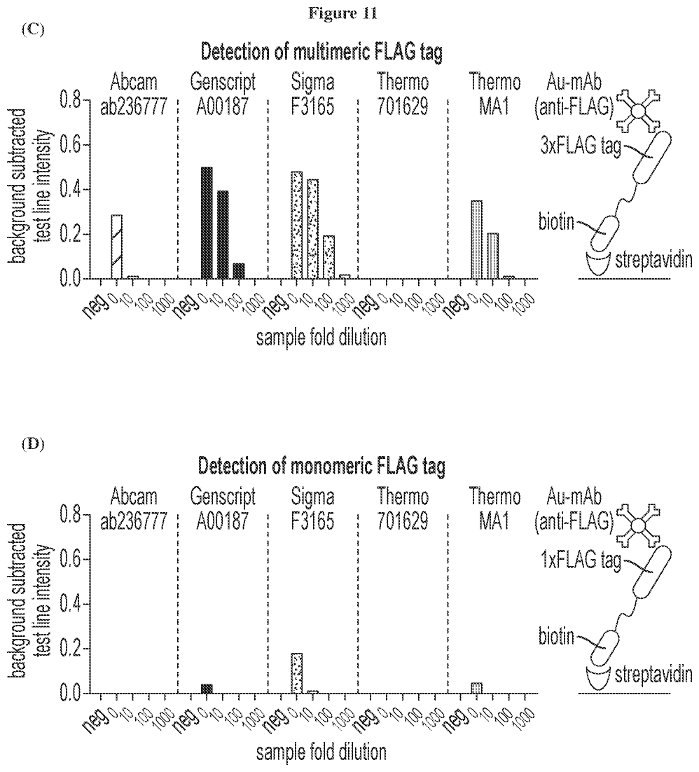

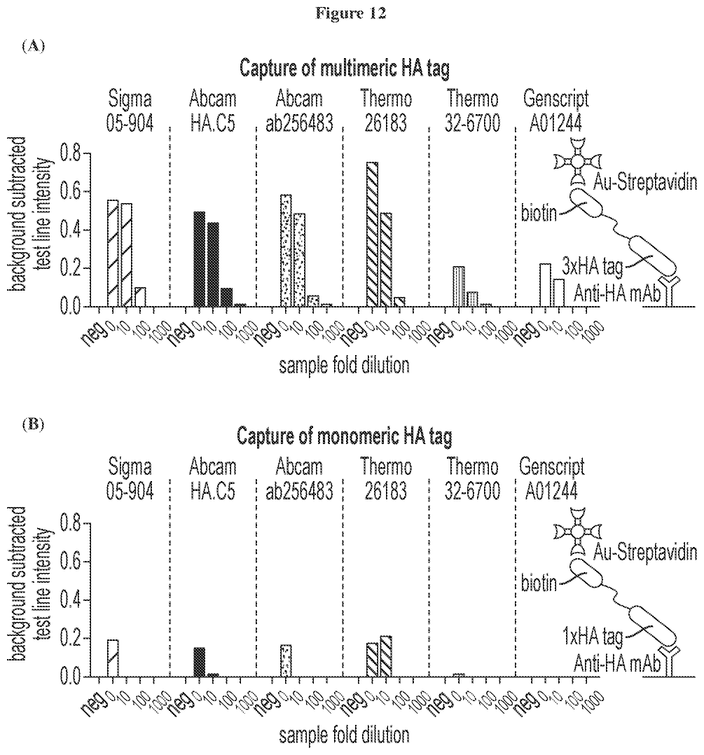

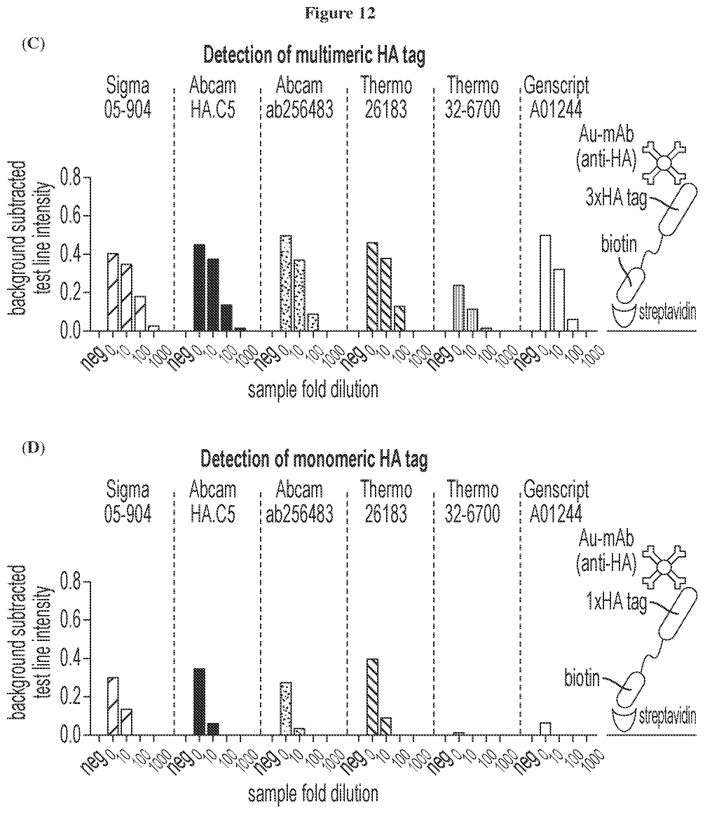

In some embodiments, the reporter is a polypeptide in which a plurality of epitopes are concatenated. In some embodiments, concatenated epitopes may improve detectable output of the disclosed system (FIG. 7-12).

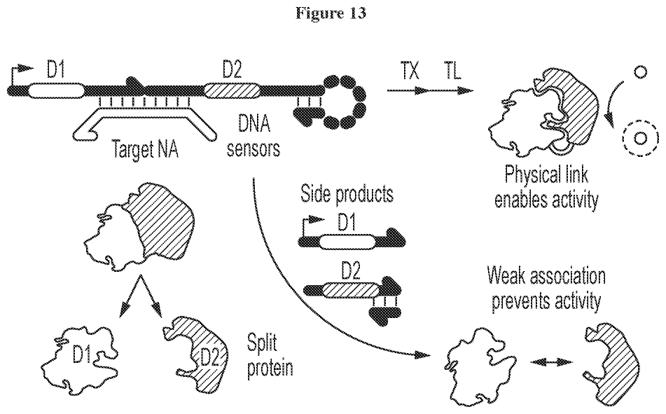

(ii) Complementing Polypeptides. In some embodiments, a reporter is a polypeptide whose functional portions are separately encoded by the reporting elements (or their complements), and whose activity, once assembled, generates or leads to generation of, a detectable signal (see. e.g., FIG. 13). In some embodiments the first and second complementing polypeptides comprise the same polypeptide sequence. In some embodiments, the first and second polypeptides comprise different polypeptide sequences.

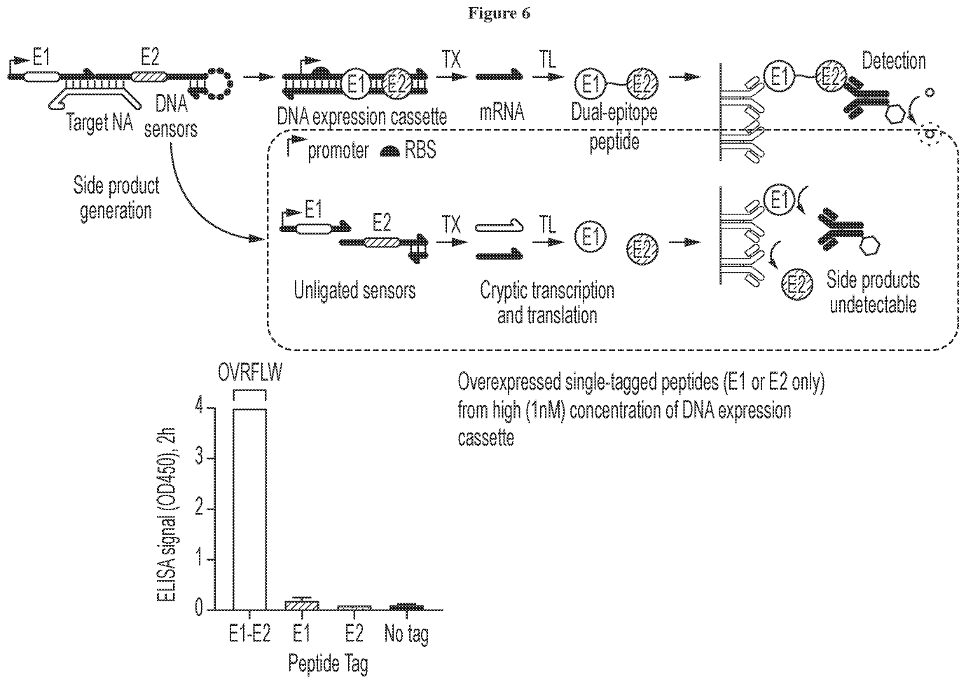

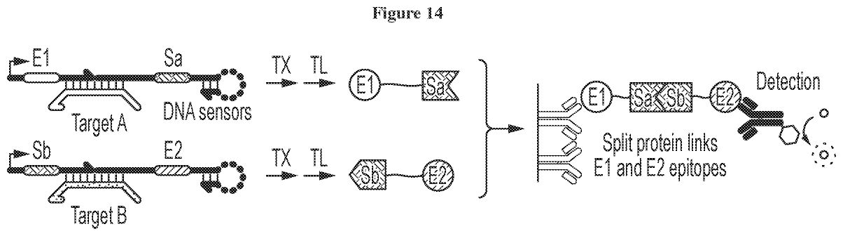

In some embodiments, the first and second complementing polypeptides must be covalently linked to enable a detectable output (FIG. 6). In some embodiments, the first and second polypeptides may be covalently linked via a second split polypeptide sequence (FIG. 14).

In some embodiments, the first and second complementing polypeptides may be non-covalently associated to enable a detectable output.

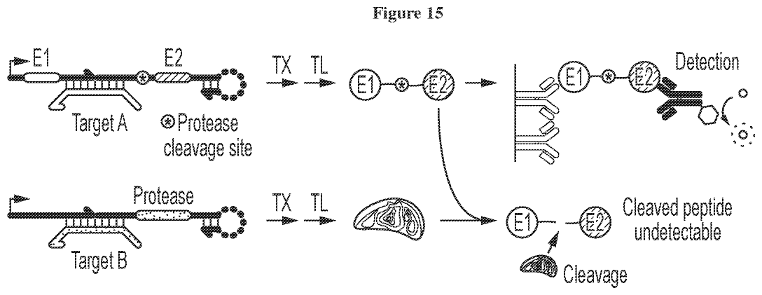

In some embodiments, an optional cleavage site is included in the linking domain between the epitope (El) resulting from the first nucleic acid sensor part and the epitope (E2) resulting from the second nucleic acid sensor part. A second target nucleic acid, Target B, expresses a protease enzyme which cleaves and deactivates the E1-E2 signal peptide (FIG. 15).

### Templating Elements

In some embodiments, a sensor part, and/or a linked nucleic acid (or its complement) includes a templating element—i.e., a sequence that directs templating of other sequences. For example, in some embodiments, a templating element may be or comprise a promoter and/or one or more transcriptional regulatory elements. Alternatively or additionally, in some embodiments, a templating element may be or comprise an origin of replication and/or a binding site for an extendible primer.

In some embodiments, a templating element may be or comprise a sequence (i.e., an “expression element”) that directs (or is the complement of one that directs) expression of associated sequences. For example, in some embodiments, an expression element is or comprises a promoter and/or one or more transcriptional regulatory elements (e.g., enhancers, repressor binding sites, etc), so that multiple RNA templates (or copies) of the ligated strand are generated by incubating, e.g., a duplex as described above, with an RNA polymerase.

In some embodiments, such an expression element may be or comprise an origin of replication, so that multiple DNA templates (or copies) of the strand are generated by incubation with a DNA polymerase.

Those of ordinary skill in the art, reading the present disclosure, will appreciate that an appropriate expression element for use in a particular embodiment may be selected to generate (or permit ready generation of - e.g., by denaturation) a templated product (e.g., RNA, ssDNA, or dsDNA) that is effective to be or generate a detectable output.

For example, FIG. 1 depicts a system in which a first nucleic acid sensor comprises an expression element (specifically, a promoter), and a first target hybridization element, and a second nucleic acid sensor comprises a second target hybridization element and a primer element. Linkage of these two nucleic acid sensor parts generates a linked strand that is copied by extension of a primer binding to the primer element (and, optionally, amplified, by extension of the same primer or a different primer together with a displaced primer that hybridizes to the opposing strand, as would be understood by those skilled in the art) or translated. Transcription from the expression element generates a transcript that is translated into a peptide antigen amenable to detection by lateral flow.

### Target Nucleic Acids

Those skilled in the art will immediately appreciate that technologies provided herein are broadly applicable to achieve detection of a wide range of nucleic acids including, for example, nucleic acids from an infectious agent (e.g., a virus, microbe, parasite, etc), nucleic acids indicative of a particular physiological state or condition (e.g., presence or state of a disease, disorder or condition such as, for example, cancer or an inflammatory or metabolic disease, disorder or condition, etc), prenatal nucleic acids, etc.

In some embodiments, a target nucleic acid includes a characteristic sequence element indicating of the relevant infectious agent or physiological state or condition; in some embodiments, a sensor system as described herein detects such characteristic sequence element. In some embodiments, a characteristic sequence element may be or comprise one or more single nucleotide polymorphisms.

In some embodiments, a target nucleic acid may be or comprise one that is a marker for presence or level of a particular infectious agent or physiological state or condition.

In many embodiments, provided technologies are particularly useful or applicable for detection of low-abundance (e.g., less than about 10 fM, or about 1 fM, or about 100 aM) nucleic acids.

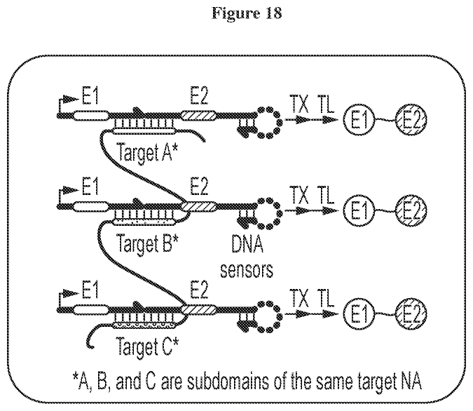

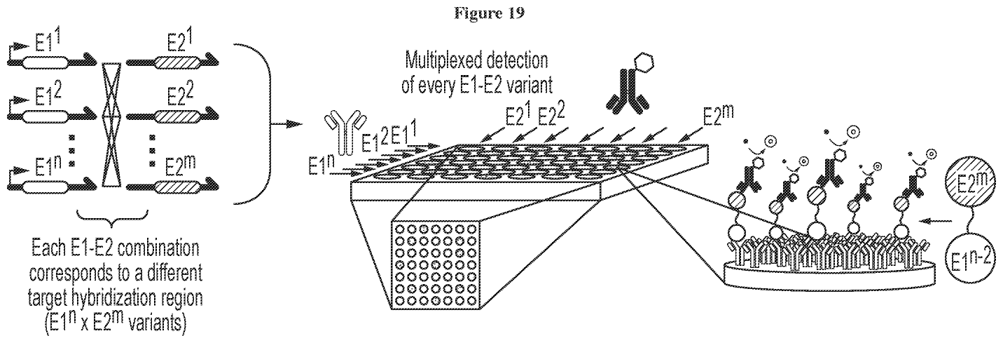

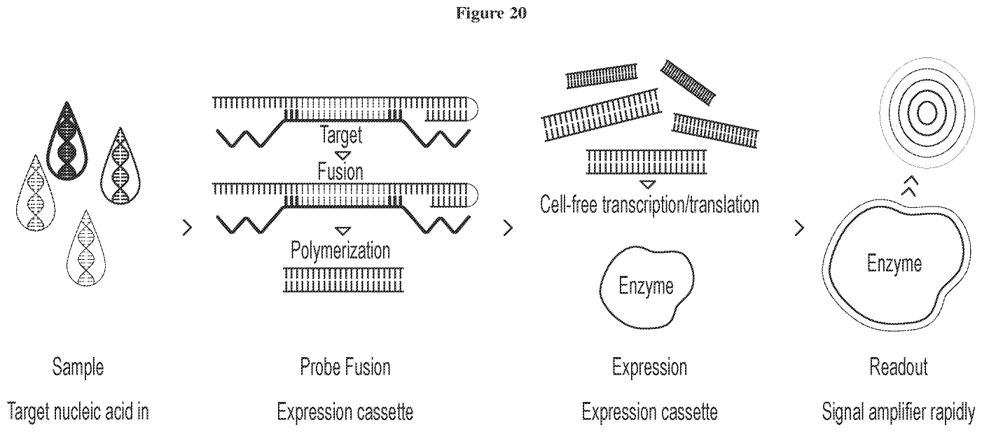

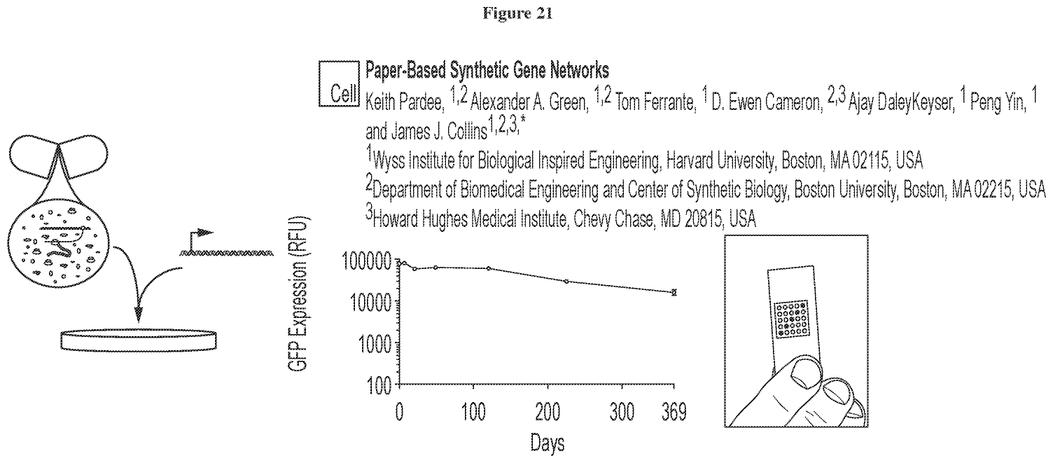

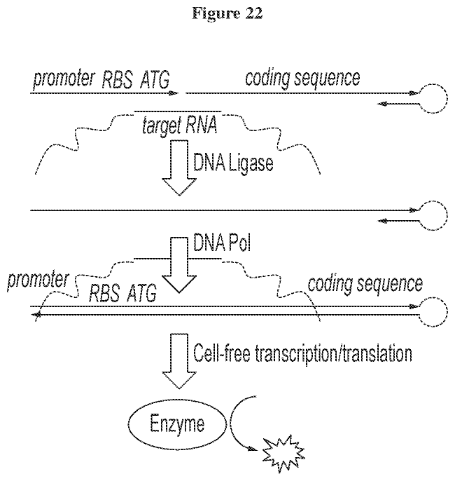

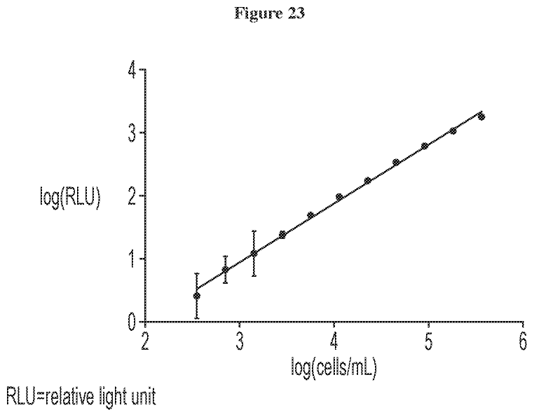

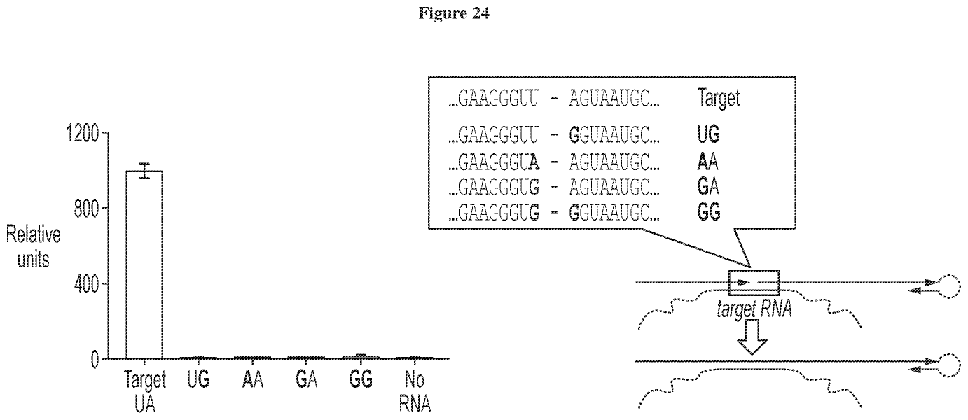

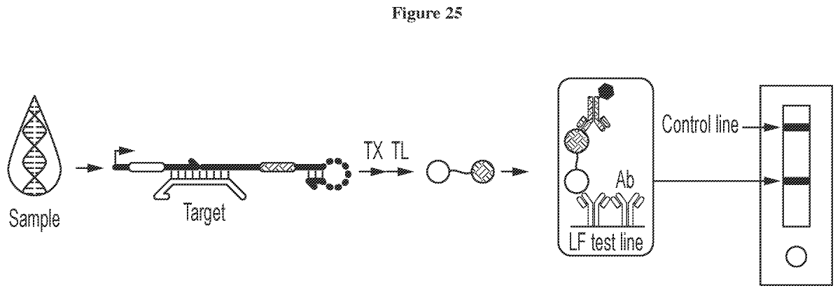

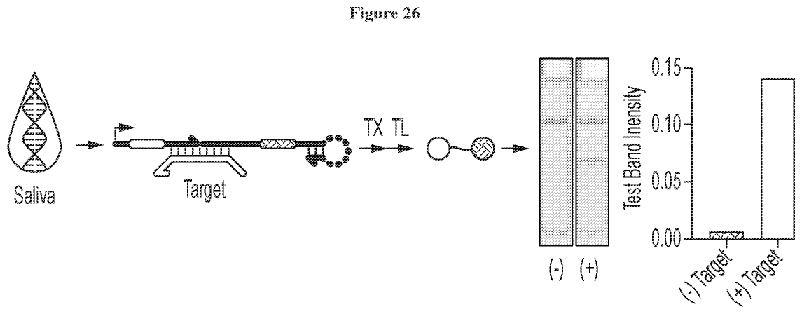

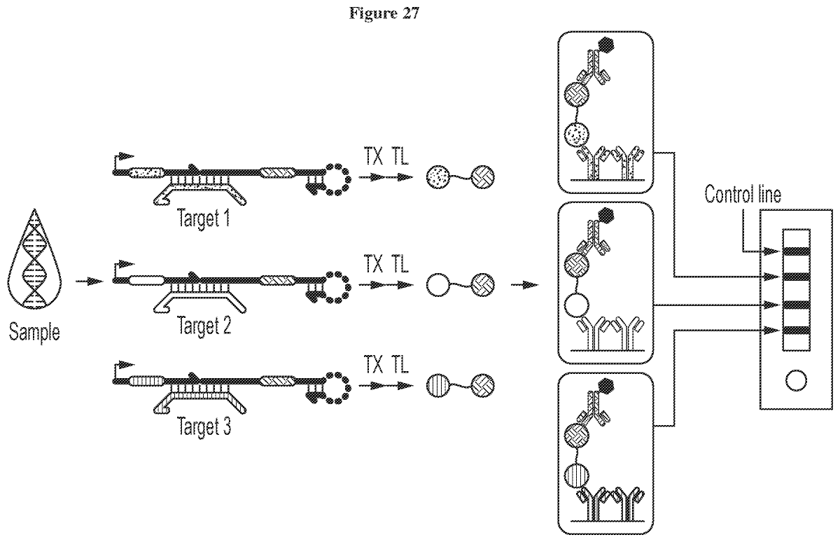

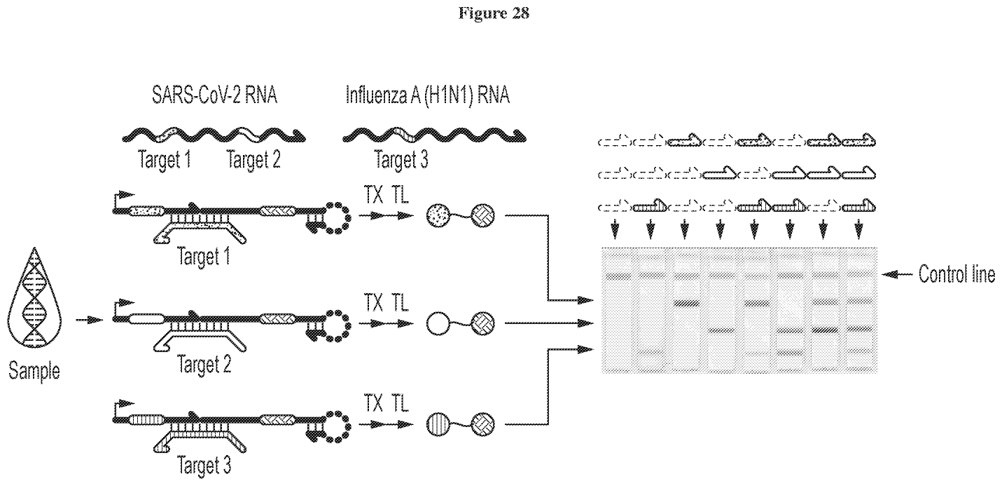

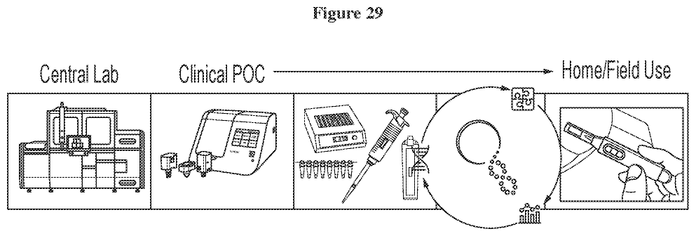

Typically, provided technologies will be applied to one or more samples to assess presence and/or level of one or more target nucleic acids in the sample. In some embodiments, the sample is a biological sample: in some embodiments, a sample is an environmental sample. In some embodiments, multiple target nucleic acids may exist within a large target nucleic acid strand (FIG. 18).

In some embodiments, a sample will be processed (e.g., nucleic acids will be partially or substantially isolated or purified out of a primary sample): in some embodiments, only minimal processing will have been performed (i.e., the sample will be a crude sample).

### Uses

Those skilled in the art, reading the present specification, will immediately appreciate that technology it provides is useful in a wide range of contexts, and can be applied in a variety of formats.

The claimed sensors can be used for in vitro detection any RNA or ssDNA (e.g., ssDNA viruses, or ssDNA generated by denaturation of genomic dsDNA), or SNPs. This enables detection of a variety of micro-organims and nucleic acids indicative of infection or other factors associated with human health, animal health, and plant health.

RNA targets include messenger RNA, microRNA, viral genomic RNA, and ribosomal RNA. A particularly useful target for bacterial detection is ribosomal RNA (e.g., 16S or 23S rRNA) as it can be present at many copies per individual bacterial cell and can be used to distinguish bacterial genus/species. Infectious bacterial target organisms include, but are not limited to species of Streptococcus, Staphylococcus, Bacillus, Campylobacter, Chlamydia, Clostridium, Enterococcus, Escherichia, Helicobacter, Listeria, Mycobacterium, Salmonella, Vibrio, Yersinia. A description of a variety of 16S ribosomal RNA target regions for the diagnosis of pathogenic bacteria is further described in, e.g., J. Microbiol Methods 2007 May 69(2): 330-339, which is incorporated herein by reference in its entirety. RNA viruses can be detected, for example, by designing sensors for regions of viral RNA genomes. Exemplary RNA viruses include but are not limited to influenza virus, sika virus, ebola virus, rotavirus, polio virus, dengue virus, yellow fever virus, hepatitis C virus, measles virus, and rabies virus.

In other embodiments, the sensor system described herein, and methods of using thereof, may be used for the detection of a target nucleic acid which differs from another nucleic acid in the sample by a single base, enabling discrimination of single nucleotide polymorphisms (SNPs). In some embodiments, the location of the SNP within the hybridization region is positioned at one of the two bases of the linking junction, on either the upstream or downstream ssDNA domain. In other embodiments, one or more mismatched bases may be additionally introduced within the hybridization region. Destabilization of the hybridization regions due to the presence of the SNP would impede the ability for the ligase to successfully link the upstream and downstream domains, resulting in a differential signal output. In various embodiments, the junction is configured to hybridize against a polymorphism of the target nucleic acid.

In some embodiments, one or more components (e.g., target nucleic acid, nucleic acid sensors, linked strand, primer(s), templating component(s) (e.g., DNA polymerase, RNA polymerase, nucleotides, etc), expression components (e.g., RNA polymerase, ribosome, nucleotides, tRNAs, etc), and combinations thereof) may be associated with (e.g., attached to) a solid support.

In some embodiments, a plurality of nucleic acid sensor sets is utilized substantially simultaneously, so that multiple target nucleic acids may be detected contemporaneously.

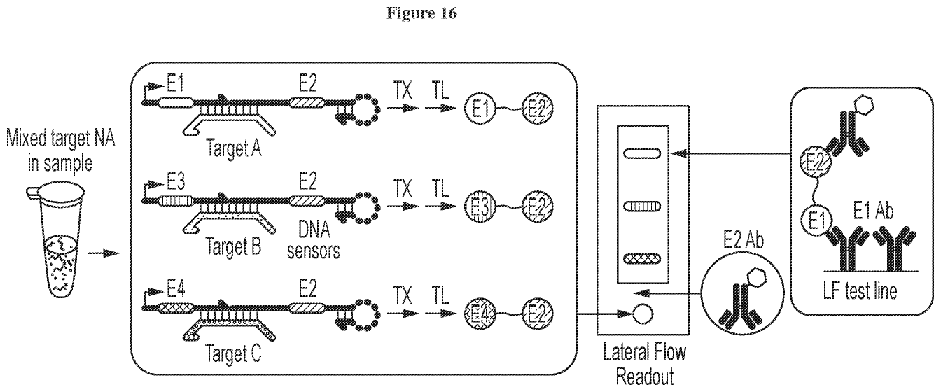

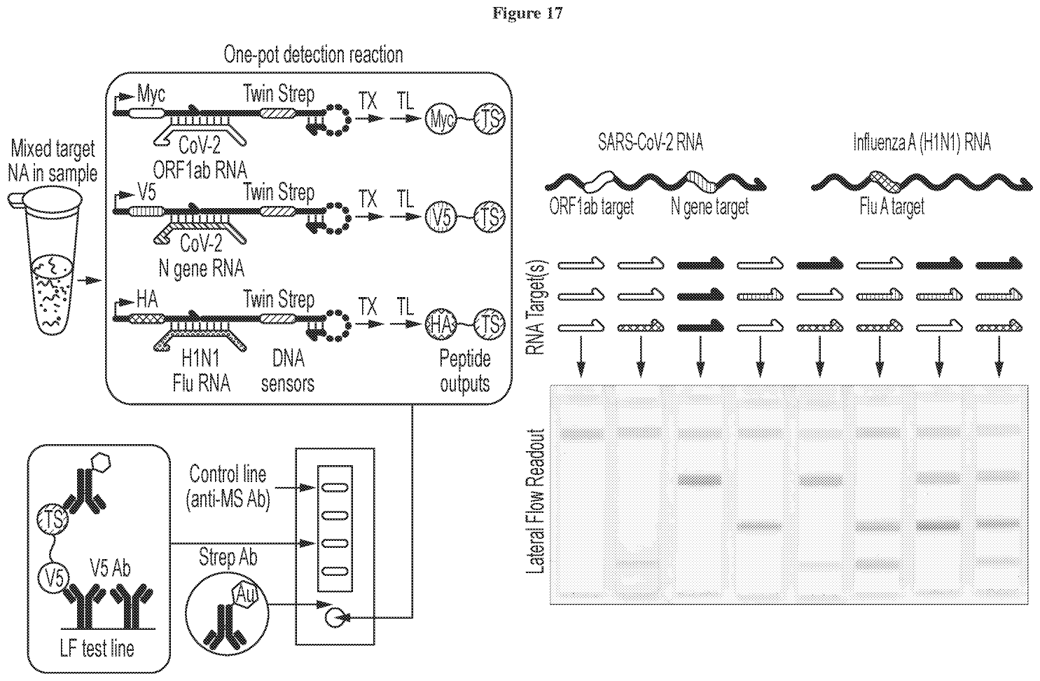

In some embodiments, provided technologies may be multiplexed, for example. Indeed, as described herein, in some embodiments, provided technologies may be particularly useful for multiplexed (e.g., simultaneous) analyses of a plurality of products amenable to lateral flow assessment (FIG. 16-17).

In some embodiments, provided technologies may be multiplexed, for example, utilizing different Cas enzymes (and/or readouts) for different target nucleic acid sequences and/or different ligation oligonucleotides.

### Exemplification

### Example 1: Overview of an INSPECTR Split Epitope System

The present example demonstrates the INSPECTR split epitope nucleic acid detection system (FIG. 1). At least two nucleic acid sensor parts hybridize to a target nucleic acid sequence, generating a double-stranded nucleic acid cassette. A first and a second nucleic acid sensor part, encodes an epitope or reporter, E1 and E2, respectively. Transcription, translation, or transcription and translation generate a juxtaposed reporter comprising the two epitope or reporter domains, E1 and E2. The linked epitope or reporter domains can subsequently be detected. To block binding of the first and second nucleic acid sensor parts to off-target nucleic acids or when utilized, capture oligos or reporter oligos, a cis-repressed hairpin structure is employed for both sensor parts. Cryptic transcription, translation, or transcription and translation resulting in unlinked epitope domains are generated as side-products that are unable to be detected (FIG. 1).

### Example 2: INSPECTR Detection May Be Coupled to Various Readout Platforms

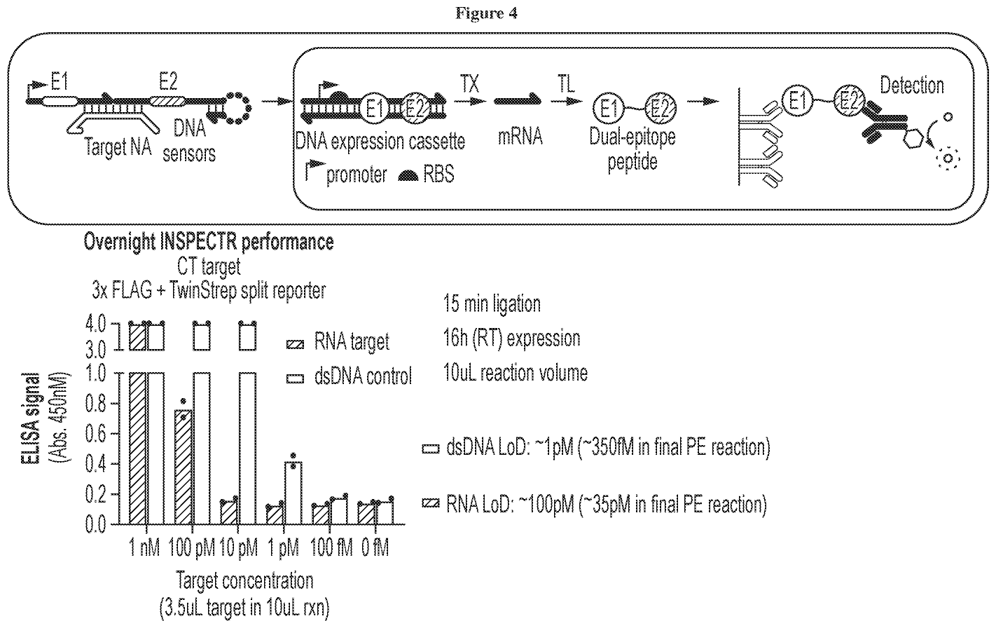

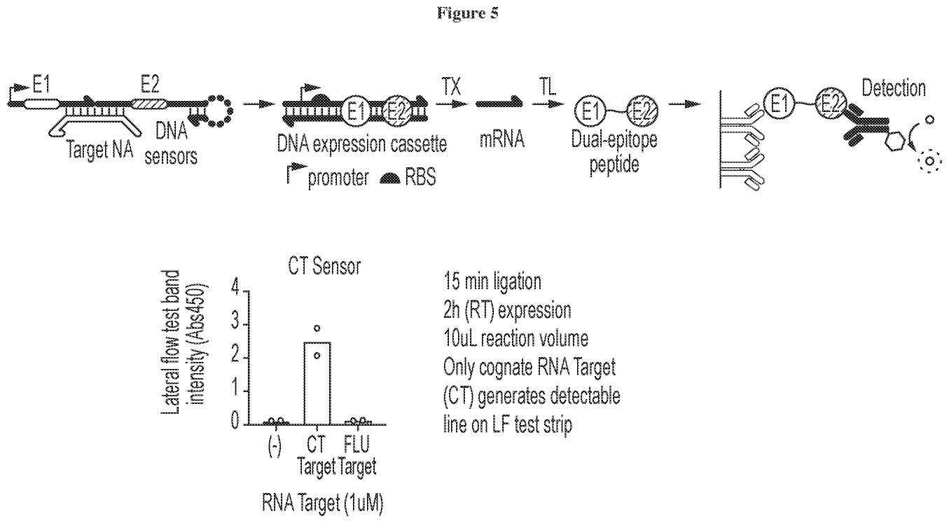

The present example demonstrates use of the INSPECTR split epitope system coupled to either an Enzyme-Linked Immunosorbent Assay (ELISA) readout (FIG. 4) or to Lateral Flow (LF) readout (FIG. 5). INSPECTR performance with a 3×FLAG×TwinStrep split reporter coupled to an ELISA readout was tested by mixing varying concentrations of the target nucleic acid, purified Chlamydia (CT) RNA, with the ligation master mix (Table 1). Purified dsDNA expressing the reporter polypeptide generated by the detection of the CT RNA was included as a positive control. The ligation reaction was incubated for 15 minutes at room temperature. Subsequently, 5 μL of a reconstituted protein synthesis composition, or cell-free system, or PURExpress master mix (Table 2) was mixed with 5 μL of the ligation reaction and incubated for 16 hours at room temperature. Reporter activity generated by detection of the CT RNA in the reaction mixture was measured by ELISA at 450 nM. The limit of detection (LoD) for CT RNA was determined as 100 μM (or 35pM in final PURExpress reaction) and the LoD for the dsDNA was determined as 1 μM (or 350fM in final PURExpress reaction) (FIG. 4).

INSPECTR performance coupled to a Lateral Flow (LF) readout was also tested. 1.5 μL of ligation master mix (Table 1) was mixed with 3.5 μL of CT RNA (1 μM) and incubated for 15 minutes at room temperature. As a negative control, sensors were incubated with no target or with a non-target RNA sequence (FLU). Subsequently, 5 μL of PURExpress master mix (Table 2) was added to the ligation reaction (10 μL final volume) and incubated for 2 hours. Following the 2 hour incubation, lateral flow test band intensity was measured at 450 nM. Little to no detectable signal was measured when the reaction was completed with no target or with the non-target RNA sequence (FIG. 5).

### Example 3: E1-E2 Epitopes Must be Linked for Detection

The present example demonstrates the E1-E2 epitopes must be linked to generate a detectable signal). Either no tag, only E1, only E2, or linked E1-E2 were overexpressed from 1 nM of DNA expression cassette and subsequently detected using an ELISA measured at 450 nM. Little to no detectable signal was measured when either no tag, only E1, or only E2 were expressed, while robust signal was detected when the linked E1-E2 epitopes were expressed, suggesting the E1-E2 epitopes must be linked for detection (FIG. 6).

### Example 4: Concentration of Epitope Tags Improved Binding Efficiency (FIG. 7-12)

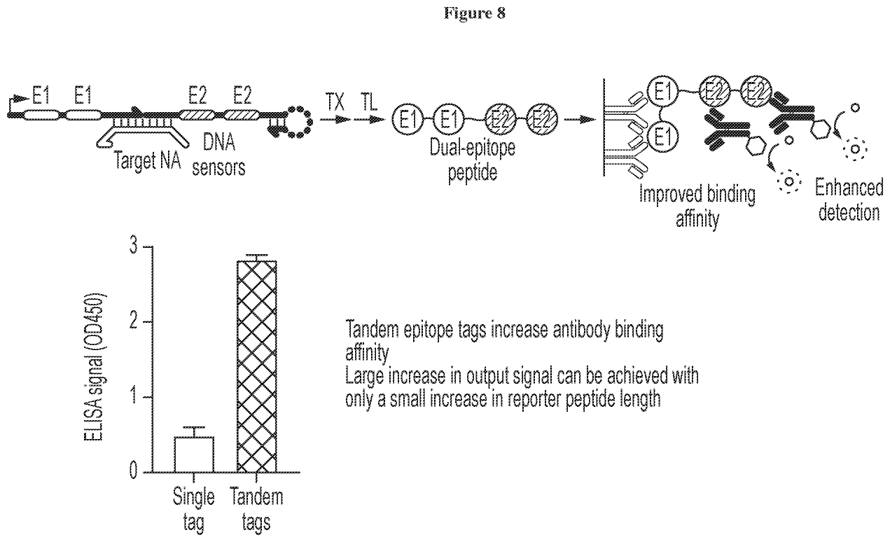

The present example demonstrates that the inclusion of tandem epitopes within either one or both nucleic acid sensor part improves output signal by increasing antibody binding. FIG. 7 demonstrates an exemplary sensor design including tandem epitopes. To test whether inclusion of tandem epitopes in each nucleic acid sensor part improved output signal, INSPECTR performance coupled to an ELISA assay was completed with a nucleic acid sensor set in which each nucleic acid sensor part comprised a tandem epitope compared to a nucleic acid sensor set in which each nucleic acid sensor part comprised a single epitope. Inclusion of tandem epitope tags in each nucleic acid sensor part increased antibody binding affinity enhancing output signal using an ELISA assay compared to the reaction using nucleic acid sensor parts comprising single epitopes (FIG. 8).

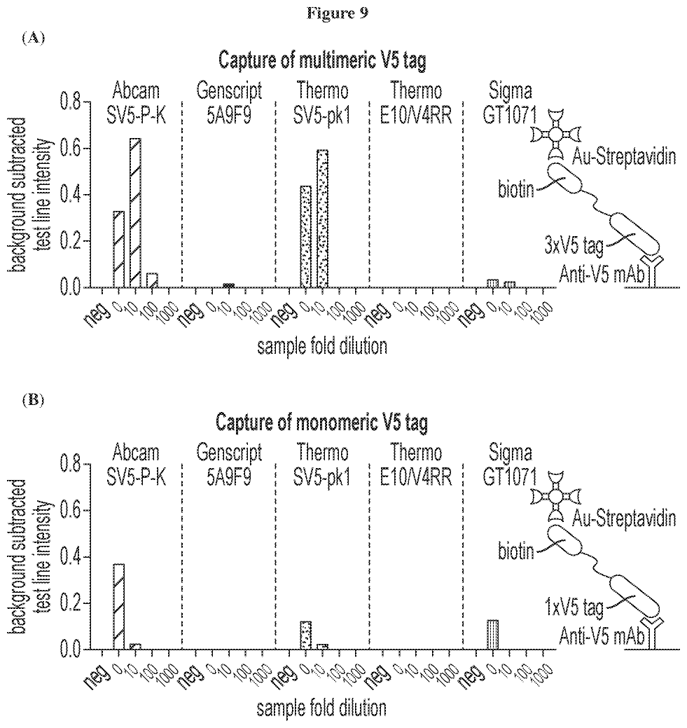

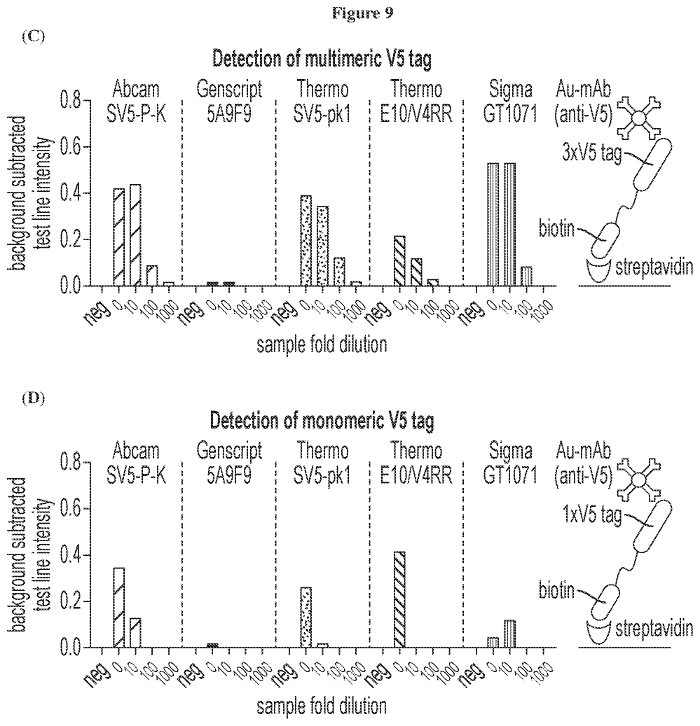

To further determine the effect of concatenated tags on capture and detection, multiple epitope tags (V5, Myc, FLAG, and HA) were tested for sensitivity by comparing sensors either comprising one or three tandem epitope tags over a range of concentrations of peptide (FIG. 9-12). Monoclonal antibodies directed against either V5, Myc, FLAG, or HA were functionalized on nitrocellulose membrane capture and detected. Peptides with 3×tags (FIG. 9-12, A) showed increased sensitivity compared to peptides with 1×tags (FIG. 9-12, B). Similarly, V5, Myc, FLAG, or HA monoclonal antibodies were functionalized on colloidal gold and detected. Peptides with 3×tags (FIG. 9-12, C) showed increased sensitivity compared to peptides with 1×tags (FIG. 9-12, D).

### Example 5 Exemplary Sequences.

The present example provides certain exemplary sequences used or described in the present disclosure.

| Name | Sequence (5′ to 3′) | Description |
|---|---|---|
| EAP0025 | GGACGGAAGTTTATAATAAGTAAGGAGGTTTTTTatgGGA | Avi-Tag- |
| | TTGAACGACATCTTCGAGGCCCAGAAAATTGAGTGGCA | 12degen- |
| | TGAGNYNNNYVNNNYNNNYVNNNYNNNYVNNNYNNNYV | 3xFLAG |
| | NNGATTATAAAGACCACGACGGTGATTATAAGGACCAC | |
| | GACATTGATTACAAAGATGATGATGATAAATAATAA | |
| EAP0026 | GAATTAATACGACTCACTATAGGGACGGAAGTTTATAAT | t7 fwd primer |
| | AAGTAAGGAGGTTTTTTatg | |
| EAP0027 | TTATTATTTATCATCATCATCTTTGTAATCAATGTCGTGG | Cterm 3x |
| | | FLAG rev |
| | | primer |
| EAP0030 | GGACGGAAGTTTATAATAAGTAAGGAGGTTTTTTatgGGA | Avi-Tag- |
| | TTGAACGACATCTTCGAGGCCCAGAAAATTGAGTGGCA | 12degen- |
| | TGAGNYNNNYVNNNYNNNYVNNNYNNNYVNNNYNNNYV | 3xHA |
| | NNTACCCCTACGATGTCCCCGACTACGCTGGGTATCCG | |
| | TATGATGTACCAGATTACGCCGGGTACCCTTACGATGT | |
| | GCCTGACT | |
| EAP0031 | TTATTACGCATAGTCAGGCACATCGTAAGGGTAC | Cterm 3x HA |
| | | rev primer |
| EAP0034 | GGACGGAAGTTTATAATAAGTAAGGAGGTTTTTTatgGGA | AviTag- |
| | TTGAACGACATCTTCGAGGCCCAGAAAATTGAGTGGCA | 12degen- |
| | TGAGNYNNNYVNNNYNNNYVNNNYNNNYVNNNYNNNYV | 3xMyc |
| | NNGAGCAAAAGTTAATCTCTGAAGAAGACCTGGAACAG | |
| | AAACTTATCTCTGAGGAAGATTTAGAGCAAAAACTGATC | |
| | TCAGAAG | |
| EAP0035 | TTATTAAAGGTCTTCTTCTGAGATCAGTTTTTGCTCTAAA | Cterm 3x |
| | TCTTCC | Myc rev |
| | | primer |
| EAP0040 | GGACGGAAGTTTATAATAAGTAAGGAGGTTTTTTatgGGA | AviTag- |
| | TTGAACGACATCTTCGAGGCCCAGAAAATTGAGTGGCA | 12degen-3x |
| | TGAGNYNNNYVNNNYNNNYVNNNYNNNYVNNNYNNNYV | V5 |
| | NNGGCAAACCCATCCCCAATCCGCTGTTAGGGCTGGAT | |
| | AGCACAGGAGGTAAGCCCATTCCTAACCCACTGTTGGG | |
| | CTTGGATA | |
| EAP0041 | TTATTAGGTTGAATCCAAACCCAGTAACGGGTTCGGGAT | Cterm 3x V5 |
| | AGGTTTACCACCAGTACTATCCAAGCCCAACAGTGGGT | rev primer |
| EAP0090 | TTATTACTTATCGTCGTCATCCTTGTAATCNNVYNNNYN | AviTag- |
| | NNVYNNNYNNNVYNNNYNNNVYNNNYNCTCATGCCACT | 12degen- |
| | CAATTTTCTGGGCCTCGAAGATGTCGTTCAATCCcatAAA | FLAG |
| | AAACCTCCTTACTTATTATAAACTTCCGTCC | |
| EAP0091 | TTATTACGCATAATCCGGAACGTCGTAGGGATANNVYNN | AviTag- |
| | NYNNNVYNNNYNNNVYNNNYNNNVYNNNYNCTCATGCC | 12degen-HA |
| | ACTCAATTTTCTGGGCCTCGAAGATGTCGTTCAATCCcat | |
| | AAAAAACCTCCTTACTTATTATAAACTTCCGTCC | |
| EAP0092 | TTATTAAAGGTCCTCCTCCGAAATCAATTTCTGTTCNNVY | AviTag- |
| | NNNYNNNVYNNNYNNNVYNNNYNNNVYNNNYNCTCATG | 12degen-Myc |
| | CCACTCAATTTTCTGGGCCTCGAAGATGTCGTTCAATCC | |
| | catAAAAAACCTCCTTACTTATTATAAACTTCCGTCC | |
| EAP0093 | TTATTATGTCGAATCCAGACCAAGAAGCGGGTTTGGAAT | AviTag- |
| | TGGTTTCCCNNVYNNNYNNNVYNNNYNNNVYNNNYNNN | 12degen-V5 |
| | VYNNNYNCTCATGCCACTCAATTTTCTGGGCCTCGAAGA | |
| | TGTCGTTCAATCCcatAAAAAACCTCCTTACTTATTATAAA | |
| | CTTCCGTCC | |
| PDC0181 | GAATTAATACGACTCACTATAGGGACGGAAGTTTATAAT | 3xFLAG-CT- |
| | AAGTAAGGAGGTTTTTTatgGATTACAAAGACCACGATGG | TwinStrep |
| | TGACTACAAAGACCATGATATAGATTACAAGGATGATGA | |
| | TGATAAAAATTATCTATGCATTACTAACCCTTCCGCCACT | |
| | AAAAGTGCGTGGTCACATCCTCAATTCGAGAAGGGCGG | |
| | CGGGAGTGGAGGTGGCTCCGGTGGATCTGCTTGGTCA | |
| | CACCCCCAATTCGAAAAATAATAA | |
| PDC0188 | GAATTAATACGACTCACTATAGGGACGGAAGTTTATAAT | SSA (3xFLAG- |
| | AAGTAAGGAGGTTTTTTatgGATTACAAAGACCACGATGG | CT) |
| | TGACTACAAAGACCATGATATAGATTACAAGGATGATGA | |
| | TGATAAAAATTATCTATGCATTACT | |
| PDC0193 | AACCCTTCCGCCACTAAAAGTGCGTGGTCACATCCTCA | SSB (CT- |
| | ATTCGAGAAGGGCGGGGGGAGTGGAGGTGGCTCCGGT | TwinStrep) |
| | GGATCTGCTTGGTCACACCCCCAATTCGAAAAATAATAA | |
| PDC0197 | TTATTATTTTTCGAATTGGGGGT | RevP |
| | | (TwinStrep) |
| CT64 | TGTTTCGGCAATTGTTTAGTGGCGGAAGGGTTAGTAATG | 64 nt |
| | CATAGATAATTTGTCCTTAACTTGG | fragment of |
| | | CT 16S |
| | | rRNA |
| PDC0143 | GAATTAATACGACTCACTATAGGGACGGAAGTTTATAAT | FLAG Tag- |
| | AAGTAAGGAGGTTTTTTatgGACTACAAAGACGATGACGA | NanoLuc |
| | CAAGGTATTTACTCTACGCGACTTCGTAGGCGATTGGC | protein- |
| | GCCAGACCGCAGGCTACAACTTAGACCAAGTCCTAGAA | StrepII Tag |
| | CAGGGCGGTGTTTCAAGCTTATTCCAAAACCTAGGAGT | |
| | AAGCGTAACGCCGATCCAACGCATTGTTCTATCGGGCG | |
| | AAAATGGCCTCAAAATCGATATTCATGTGATCATTCCAT | |
| | ACGAGGGGTTATCCGGAGACCAGATGGGACAGATCGA | |
| | AAAGATCTTCAAAGTAGTCTACCCCGTGGACGATCACCA | |
| | TTTCAAGGTTATCCTTCATTATGGAACGCTTGTCATCGA | |
| | CGGGGTCACCCCGAACATGATTGACTACTTTGGTCGTC | |
| | CTTATGAAGGAATTGCGGTCTTTGACGGGAAGAAGATTA | |
| | CTGTTACAGGGACCCTTTGGAACGGTAATAAAATCATTG | |
| | ACGAGCGTCTGATCAACCCCGATGGAAGTCTTCTTTTTC | |
| | GTGTGACTATCAACGGAGTGACTGGTTGGCGCCTGTGT | |
| | GAACGCATTCTTGCTAACTGGAGTCACCCCCAATTTGAA | |
| | AAAtaataa | |
| PDC0154 | GAATTAATACGACTCACTATAGGGACGGAAGTTTATAAT | FLAG Tag- |
| | AAGTAAGGAGGTTTTTTatgGACTACAAAGACGATGACGA | NanoLuc |
| | CAAGGTATTTACTCTACGCGACTTCGTAGGCGATTGGC | protein |
| | GCCAGACCGCAGGCTACAACTTAGACCAAGTCCTAGAA | |
| | CAGGGCGGTGTTTCAAGCTTATTCCAAAACCTAGGAGT | |
| | AAGCGTAACGCCGATCCAACGCATTGTTCTATCGGGCG | |
| | AAAATGGCCTCAAAATCGATATTCATGTGATCATTCCAT | |
| | ACGAGGGGTTATCCGGAGACCAGATGGGACAGATCGA | |
| | AAAGATCTTCAAAGTAGTCTACCCCGTGGACGATCACCA | |
| | TTTCAAGGTTATCCTTCATTATGGAACGCTTGTCATCGA | |
| | CGGGGTCACCCCGAACATGATTGACTACTTTGGTCGTC | |
| | CTTATGAAGGAATTGCGGTCTTTGACGGGAAGAAGATTA | |
| | CTGTTACAGGGACCCTTTGGAACGGTAATAAAATCATTG | |
| | ACGAGCGTCTGATCAACCCCGATGGAAGTCTTCTTTTTC | |
| | GTGTGACTATCAACGGAGTGACTGGTTGGCGCCTGTGT | |
| | GAACGCATTCTTGCTtaataa | |
| PDC0155 | GAATTAATACGACTCACTATAGGGACGGAAGTTTATAAT | NanoLuc |
| | AAGTAAGGAGGTTTTTTatgGTATTTACTCTACGCGACTT | protein- |
| | CGTAGGCGATTGGCGCCAGACCGCAGGCTACAACTTAG | Strepll Tag |
| | ACCAAGTCCTAGAACAGGGCGGTGTTTCAAGCTTATTC | |
| | CAAAACCTAGGAGTAAGCGTAACGCCGATCCAACGCAT | |
| | TGTTCTATCGGGCGAAAATGGCCTCAAAATCGATATTCA | |
| | TGTGATCATTCCATACGAGGGGTTATCCGGAGACCAGA | |
| | TGGGACAGATCGAAAAGATCTTCAAAGTAGTCTACCCC | |
| | GTGGACGATCACCATTTCAAGGTTATCCTTCATTATGGA | |
| | ACGCTTGTCATCGACGGGGTCACCCCGAACATGATTGA | |
| | CTACTTTGGTCGTCCTTATGAAGGAATTGCGGTCTTTGA | |
| | CGGGAAGAAGATTACTGTTACAGGGACCCTTTGGAACG | |
| | GTAATAAAATCATTGACGAGCGTCTGATCAACCCCGATG | |
| | GAAGTCTTCTTTTTCGTGTGACTATCAACGGAGTGACTG | |
| | GTTGGCGCCTGTGTGAACGCATTCTTGCTAACTGGAGT | |
| | CACCCCCAATTTGAAAAAtaataa | |
| PDC0156 | GAATTAATACGACTCACTATAGGGACGGAAGTTTATAAT | NanoLuc |
| | AAGTAAGGAGGTTTTTTatgGTATTTACTCTACGCGACTT | protein (no |
| | CGTAGGCGATTGGCGCCAGACCGCAGGCTACAACTTAG | epitope tag) |
| | ACCAAGTCCTAGAACAGGGCGGTGTTTCAAGCTTATTC | |
| | CAAAACCTAGGAGTAAGCGTAACGCCGATCCAACGCAT | |
| | TGTTCTATCGGGCGAAAATGGCCTCAAAATCGATATTCA | |
| | TGTGATCATTCCATACGAGGGGTTATCCGGAGACCAGA | |
| | TGGGACAGATCGAAAAGATCTTCAAAGTAGTCTACCCC | |
| | GTGGACGATCACCATTTCAAGGTTATCCTTCATTATGGA | |
| | ACGCTTGTCATCGACGGGGTCACCCCGAACATGATTGA | |
| | CTACTTTGGTCGTCCTTATGAAGGAATTGCGGTCTTTGA | |
| | CGGGAAGAAGATTACTGTTACAGGGACCCTTTGGAACG | |
| | GTAATAAAATCATTGACGAGCGTCTGATCAACCCCGATG | |
| | GAAGTCTTCTTTTTCGTGTGACTATCAACGGAGTGACTG | |
| | GTTGGCGCCTGTGTGAACGCATTCTTGCTtaataa | |
| PDC0157 | GAATTAATACGACTCACTATAGGGACGGAAGTTTATAAT | 3x FLAG Tag- |
| | AAGTAAGGAGGTTTTTTatgGATTACAAAGACCACGATGG | NanoLuc |
| | TGACTACAAAGACCATGATATAGATTACAAGGATGATGA | protein- |
| | TGATAAAGTATTTACTCTACGCGACTTCGTAGGCGATTG | TwinStrep |
| | GCGCCAGACCGCAGGCTACAACTTAGACCAAGTCCTAG | Tag |
| | AACAGGGCGGTGTTTCAAGCTTATTCCAAAACCTAGGA | |
| | GTAAGCGTAACGCCGATCCAACGCATTGTTCTATCGGG | |
| | CGAAAATGGCCTCAAAATCGATATTCATGTGATCATTCC | |
| | ATACGAGGGGTTATCCGGAGACCAGATGGGACAGATCG | |
| | AAAAGATCTTCAAAGTAGTCTACCCCGTGGACGATCACC | |
| | ATTTCAAGGTTATCCTTCATTATGGAACGCTTGTCATCG | |
| | ACGGGGTCACCCCGAACATGATTGACTACTTTGGTCGT | |
| | CCTTATGAAGGAATTGCGGTCTTTGACGGGAAGAAGAT | |
| | TACTGTTACAGGGACCCTTTGGAACGGTAATAAAATCAT | |
| | TGACGAGCGTCTGATCAACCCCGATGGAAGTCTTCTTTT | |
| | TCGTGTGACTATCAACGGAGTGACTGGTTGGCGCCTGT | |
| | GTGAACGCATTCTTGCTAGTGCGTGGTCACATCCTCAAT | |
| | TCGAGAAGGGCGGCGGGAGTGGAGGTGGCTCCGGTG | |
| | GATCTGCTTGGTCACACCCCCAATTCGAAAAAtaataa | |
| PDC0294 | CUGCUUCAGACACUUAUGCCUGUUGGCAUCAUUCUAU | SARS-CoV-2 |
| | UGGAUUUGAUUACGUCUAUAAUCCGUU | RNA (Orf1ab, |
| | | 64 nt) |
| PDC0295 | UCGUUCCUCAUCACGUAGUCGCAACAGUUCAAGAAAU | SARS-CoV-2 |
| | UCAACUCCAGGCAGCAGUAGGGGAACU | RNA (N |
| | | gene, 64 nt) |
| PDC0291 | CAUCCGAUCACAAUUGGAAAAUGUCCAAAAUAUGUAAA | FLUA2 RNA |
| | AAGCACAAAAUUGAGACUGGCCACAG | (64 nt) |
| PDC0331 | GAATTAATACGACTCACTATAGGGACGGAAGTTTATAAT | 3xMyc- |
| | AAGTAAGGAGGTTTTTTatgGAGCAGAAACTTATTTCGGA | Orf1ab- |
| | GGAAGACTTGGAGCAAAAACTGATCAGTGAGGAAGACT | TwinStrep |
| | TAGAACAGAAGTTAATCAGTGAGGAAGATCTGCGGATTA | SSA |
| | TAGACGTAATC | |
| PDC0332 | /5Phos/AAATCCAATAGAATGATGAGTGCGTGGTCACATC | 3xMyc- |
| | CTCAATTCGAGAAGGGCGGCGGGAGTGGAGGTGGCTC | Orf1ab- |
| | CGGTGGATCTGCTTGGTCACACCCCCAATTCGAAAAAT | TwinStrep |
| | AATAA | ssB |
| PDC0334 | GAATTAATACGACTCACTATAGGGACGGAAGTTTATAAT | 2xV5-N- |
| | AAGTAAGGAGGTTTTTTatgGGCAAGCCTATCCCCAATCC | TwinStrep |
| | GCTGTTGGGACTGGATAGCACTGGTGGGAAACCTATCC | SSA |
| | CGAACCCACTTCTTGGCTTGGACTCCACCGGGCTGGAG | |
| | TTGAATTTCTTG | |
| PDC0335 | /5Phos/AACTGTTGCGACTACGTGAGTGCGTGGTCACAT | 2xV5-N- |
| | CCTCAATTCGAGAAGGGCGGCGGGAGTGGAGGTGGCT | TwinStrep |
| | CCGGTGGATCTGCTTGGTCACACCCCCAATTCGAAAAA | ssB |
| | TAATAA | |
| PDC0319 | GAATTAATACGACTCACTATAGGGACGGAAGTTTATAAT | 3xHA- |
| | AAGTAAGGAGGTTTTTTatgTACCCCTATGATGTTCCTGA | FLUA2- |
| | CTATGCTGGGTATCCTTACGATGTCCCCGACTACGCCG | TwinStrep |
| | GGTATCCGTATGACGTACCTGATTATGCTAATTTTGTGC | SSA |
| | TTTTTACA | |
| PDC0320 | /5Phos/TATTTTGGACATTTTCCAAGTGCGTGGTCACATC | 3xHA- |
| | CTCAATTCGAGAAGGGCGGCGGGAGTGGAGGTGGCTC | FLUA2- |
| | CGGTGGATCTGCTTGGTCACACCCCCAATTCGAAAAAT | TwinStrep |
| | AATAA | SSB |

### Example 6 Gap Filling Oligonucleotides

The present example provides data confirming the utility and effectiveness of bridging oligonucleotides also referred to as gap filling oligonucleotides (GFO). The present disclosure provides the observation that “ON-target” ligation of nucleic acid sensor parts is intended to occur only when two sensor parts are brought together (splinted) on a nucleic acid target (e.g., RNA or DNA). However, promiscuous activity of ligase enzymes can result in low but non-zero levels of ligation in the absence of target nucleic acid. This is challenging in the context of generating a molecular diagnostic, because this low-level ligation can be detected as a weak positive signal.

The present example demonstrates that addition of one or more “Gap-filling oligos” (GFOs) reduces the effect of mis-ligation by requiring scaffolding of additional probe strands on the nucleic acid target followed by multiple ligation events. Mis-ligation events, which are predominately single-ligation events where the GFO is not incorporated, generate a different sequence from the correct ligation product. This sequence difference then makes it possible to discriminate ON-target ligation products (generated via interaction with the nucleic acid target) from OFF-target ligation products (generated via off-target ligation).

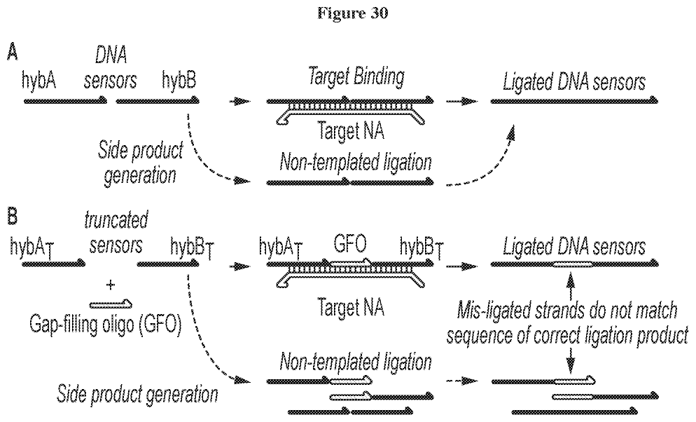

As demonstrated by FIG. 30, ends of DNA probes (e.g., nucleic acid sensor parts “hybA” and “hybB”) are moved apart from one another along the target nucleic acid, resulting in a gap between the two ends when a target is bound (“hybAT” and “hybBT”). An additional DNA strand (the gap-filling oligo) bridges the gap between these truncated ends. Two separate ligation events (on either end of the GFO) then occur to generate a completed probe reporter. This requirement for multiple ligation events (in a specified order) makes the probability of generating the correct reporter in the absence of target nucleic acid very low. The length of the GFO can be short (˜6 nt) up to very long (>100 nt), only bounded by the thermodynamic limits of the GFO to bind the target nucleic acid.

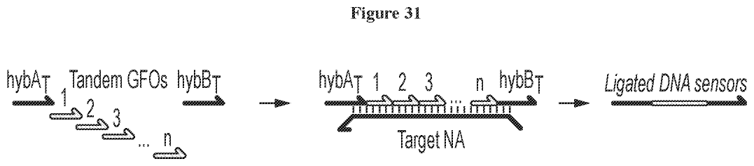

One or more GFOs are used to bridge a relatively longer gap. A higher number of GFOs generates a higher stringency by requiring more ligation events (e.g., in a specific order) to generate a correct reporter. See, for example, FIG. 31.

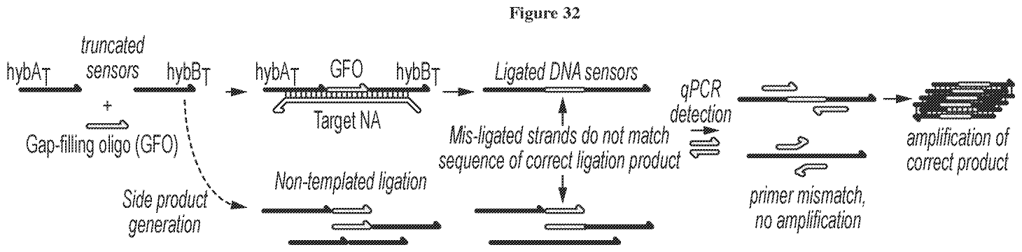

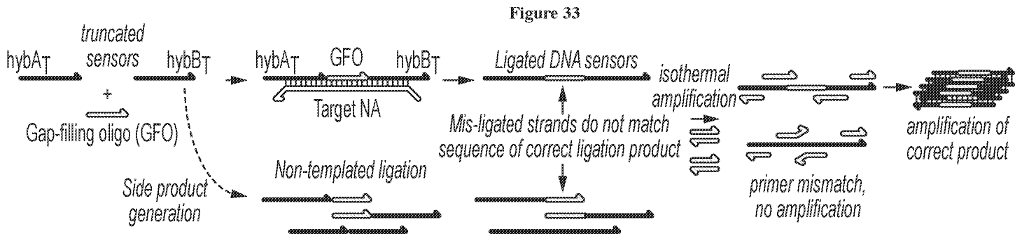

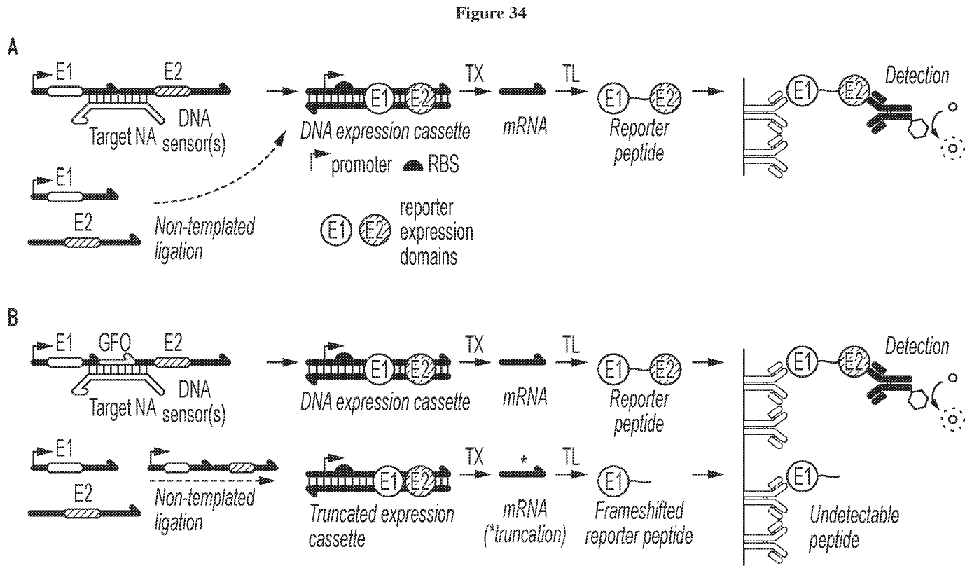

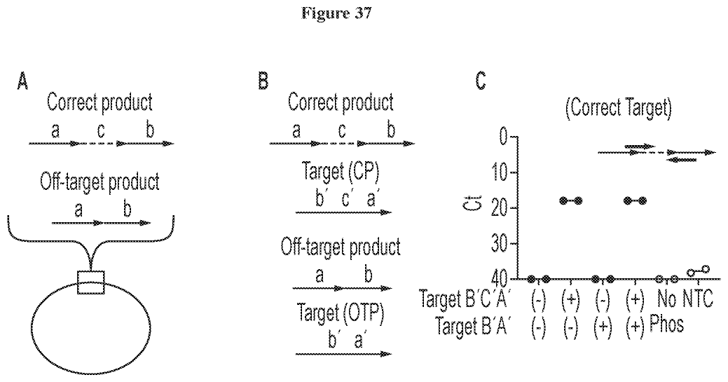

A reporter element generated by ligation can be linear or circular. That is, hybAT and hybBT (nucleic acid sensor parts) may be either trans-interacting (subdomains of separate strands) or cis-interacting (the 5′ and 3′ ends of the same ssDNA strand, forming a circular product). In turn, a reporter element can encode a substrate for several outputs. In each case, sequence differences between correctly- and incorrectly-ligated targets can be used to specifically detect only ON-target ligation events. ON- target ligation events can be specifically detected using various methods of amplification. ON- target ligation events are detected by qPCR. qPCR primers or probe(s) span the GFO region thus only amplicons with the GFO inserted are amplified and detected. See, for example, FIGS. 32 and 37. Additionally, as seen in FIG. 33, isothermal amplification techniques (e.g., rolling circle amplification, nicking enzyme amplification, etc.) detect ON-target ligation events. Amplification primers are designed within/spanning the GFO sequence, preferentially amplifying only sequences with correct GFO insertion.

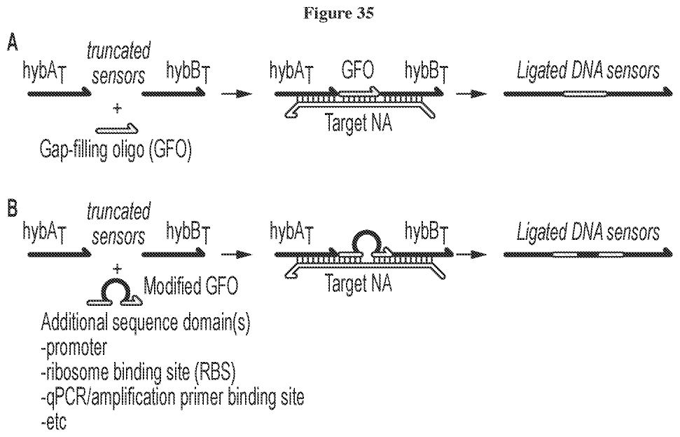

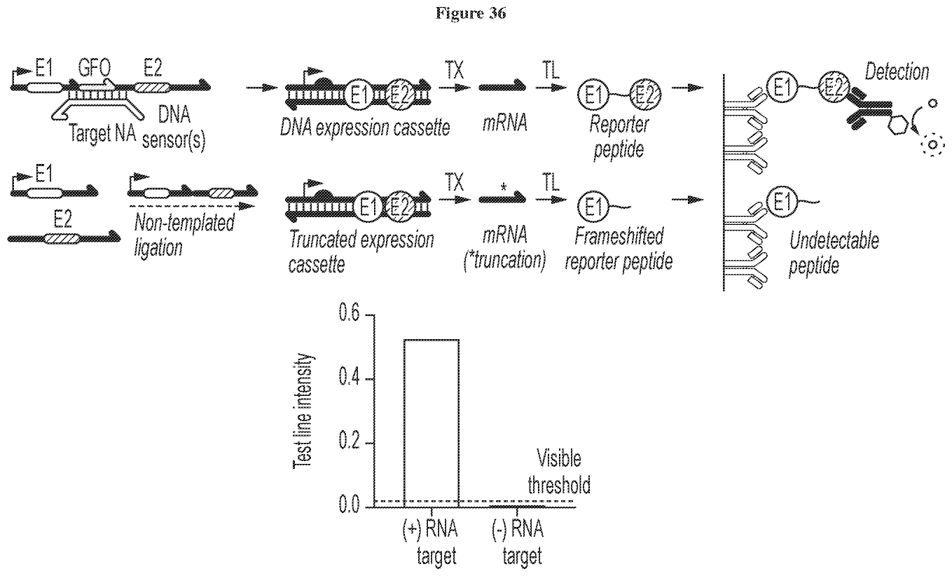

Nucleic acid sensor parts and GFOs are designed to generate reporter elements. Sensor systems as described herein generate a reporter from the reporting elements and the reporter is a polypeptide. The polypeptide is generated by the transcription and translation of a nucleic acid that results from an ON-target ligation event. GFOs can be designed of particular length such that presence of a GFO in an ON-target ligation event results in a nucleic acid comprising a proper open reading frame to produce a polypeptide reporter. The length of the GFO can be designed to be not a multiple of 3 (ex 11 nt or 13 nt but not 12 nt). In this event, a mis-ligation (ligation without the GFO) generates a frameshifted peptide which is therefore non-functional as a reporter. See, for example, FIG. 36.

In the event of a mis-ligation which puts the reporter peptide out of frame, the sequence of nucleic acid sensor parts can be designed such that a stop codon is inserted to prevent translation of the out-of-frame peptide to reduce the likelihood that additional, random amino acids are continuing to be translated. Multiple stop codons can also be added, further improving the ability to terminate translation. When the GFO is correctly ligated, this shifts the frame and removes the stop codon(s), ensuring full translation of the correct reporter output.

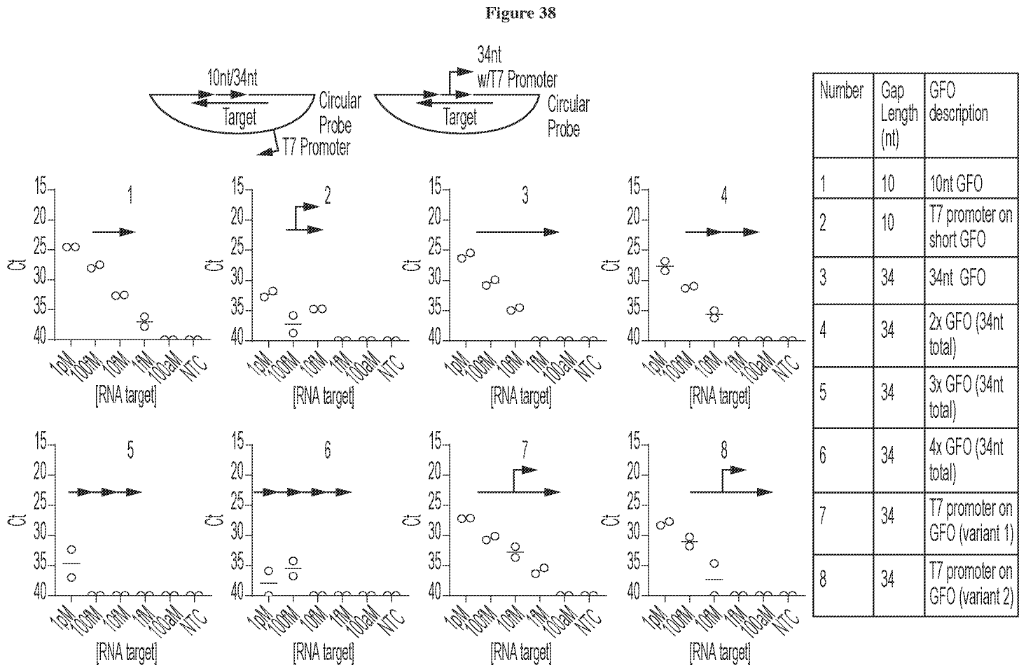

Further, as demonstrated in FIGS. 35, a GFO can contain additional inserted sequence (forming a three-way junction or other structure within the GFO binding region) For example, if the GFO is being used for INSEPCTR to generate a circularized expression cassette, a T7 promoter can be moved within a GFO. Therefore, mis-ligated probes (circular backbone without a GFO) contain both a frameshifted reporter peptide and lack a T7 promoter. See, for example, FIG. 38.

### Example 7: Exemplary Detection of a Plurality of Sub-regions Within a Larger Target Nucleic Acid

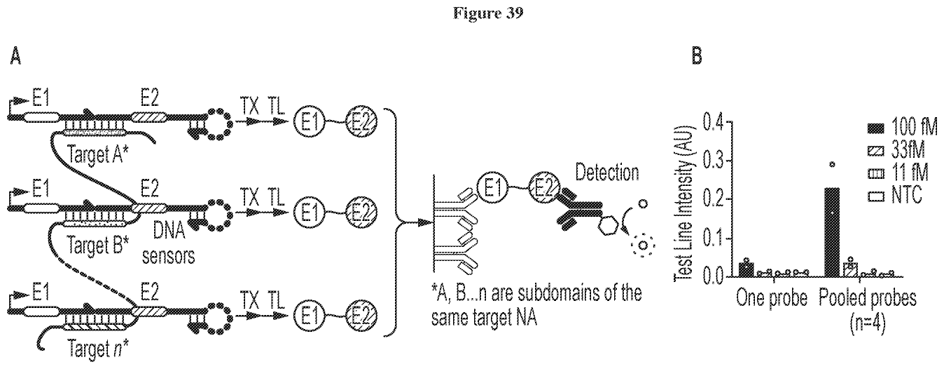

The present example provides further exemplary demonstration of detection of multiple subdomains within a larger nucleic acid strand (e.g., FIG. 18, FIG. 39). Without wishing to be bound by any one theory, a plurality of first and second nucleic acid sensor parts can be utilized targeting sub-regions (e.g., subdomains) of the same target nucleic acid to increase sensitivity of detection. In this example, circular probes utilizing gap-filling oligos (GFOs) were used (e.g., as shown in FIG. 43C). Probe(s) and GFO(s) were ligated by SplintR ligase in the presence of SARS-COV-2 genomic RNA. Polymerization by Phi29 DNA polymerase was then carried out to form double-stranded DNA product, followed by expression in a cell-free extract, followed by peptide detection using lateral flow half-strips. The entire process was carried out a 22° C. Four sets of first and second nucleic acid sensor parts targeting different regions of the same target nucleic acid, a SARS-COV-2 sequence, (“pooled probes”) show increased signal compared to a detection assay utilizing only a single set of first and second nucleic acid sensor parts (“single probe”, PDC1211, Table 7.1) selected from the set of four (FIG. 39B). PDC1211 was selected, as it was the best-performing individual probe from a broader screen of probe detection regions For the pooled probes (n=4 target regions of the SARS-COV-2 genome), the total concentration of probe was matched to the probe concentration of the single probe condition. Sequences of all probes utilized in Example 7 are summarized in Table 7.1.

TABLE 7.1
Exemplary probe sequences.

| Name | Sequence (5′ to 3′) | Description |
|---|---|---|
| PDC1205 | /5Phos/TACCAGCCTGTACGTAATAAGTAATAGTGCGTG | Probe targeting |
| | GTCACATCCTCAATTCGAGAAGGGCGGCGGGAGTGGAGG | region A of SARS- |
| | TGGCTCCGGTGGATCTGCTTGGTCACACCCCCAATTCGAA | CoV-2 RNA |
| | AAATAATAAAGGGAAGTTTATAATAAGTAAGGAGGTTT | |
| | TTTATGGATTACAAAGACCACGATGGTGACTACAAAGAC | |
| | CATGATATAGATTACAAGGATGATGATGATAAAAAGTAC | |
| | ACAATTT | |
| PDC1207 | /5Phos/GGCAGGCACTTCTGTAATAAGTAATAGTGCGTG | Probe targeting |
| | GTCACATCCTCAATTCGAGAAGGGCGGCGGGAGTGGAGG | region B of SARS- |
| | TGGCTCCGGTGGATCTGCTTGGTCACACCCCCAATTCGAA | CoV-2 RNA |
| | AAATAATAAAGGGAAGTTTATAATAAGTAAGGAGGTTT | |
| | TTTATGGATTACAAAGACCACGATGGTGACTACAAAGAC | |
| | CATGATATAGATTACAAGGATGATGATGATAAATTAGCA | |
| | GCATCTA | |
| PDC1211 | /5Phos/AAGATCTGAATCGGTAATAAGTAATAGTGCGTG | Probe targeting |
| | GTCACATCCTCAATTCGAGAAGGGCGGCGGGAGTGGAGG | region C of SARS- |
| | TGGCTCCGGTGGATCTGCTTGGTCACACCCCCAATTCGAA | CoV-2 |
| | AAATAATAAAGGGAAGTTTATAATAAGTAAGGAGGTTT | |
| | TTTATGGATTACAAAGACCACGATGGTGACTACAAAGAC | |
| | CATGATATAGATTACAAGGATGATGATGATAAAGCACAA | |
| | TCACCAA | |
| PDC1212 | /5Phos/CGATGAGAGATTCGTAATAAGTAATAGTGCGTG | Probe targeting |
| | GTCACATCCTCAATTCGAGAAGGGCGGCGGGAGTGGAGG | region D of SARS- |
| | TGGCTCCGGTGGATCTGCTTGGTCACACCCCCAATTCGAA | CoV-2 |
| | AAATAATAAAGGGAAGTTTATAATAAGTAAGGAGGTTT | |
| | TTTATGGATTACAAAGACCACGATGGTGACTACAAAGAC | |
| | CATGATATAGATTACAAGGATGATGATGATAAACCATGG | |
| | CCATTTT | |
| PDC1217 | /5Phos/TGCATAGAATGTCCAATCTAATACGACTCACTA | Gap-filling oligo |
| | TAGGGAACCCTGAGTTGAACAT | targeting region A |
| | | of SARS-CoV-2 |
| | | RNA |
| PDC1219 | /5Phos/CAGCAAAAGCACAGAAACTAATACGACTCACTA | Gap-filling oligo |
| | TAGGGGATAATACAGTTGAATT | targeting region B |
| | | of SARS-CoV-2 |
| | | RNA |
| PDC1223 | /5Phos/TCAAAGTTGAATCTGCACTAATACGACTCACTA | Gap-filling oligo |
| | TAGGGTCAGAGACAAAGTCATT | targeting region C |
| | | of SARS-CoV-2 |
| | | RNA |
| PDC1224 | /5Phos/ATATACTGCTCATACTTCTAATACGACTCACTA | Gap-filling oligo |
| | TAGGGTCCAAGTTCTTGGAGAT | targeting region D |
| | | of SARS-CoV-2 |
| | | RNA |

### Example 8: Exemplary Use of Circular Probe Configuration

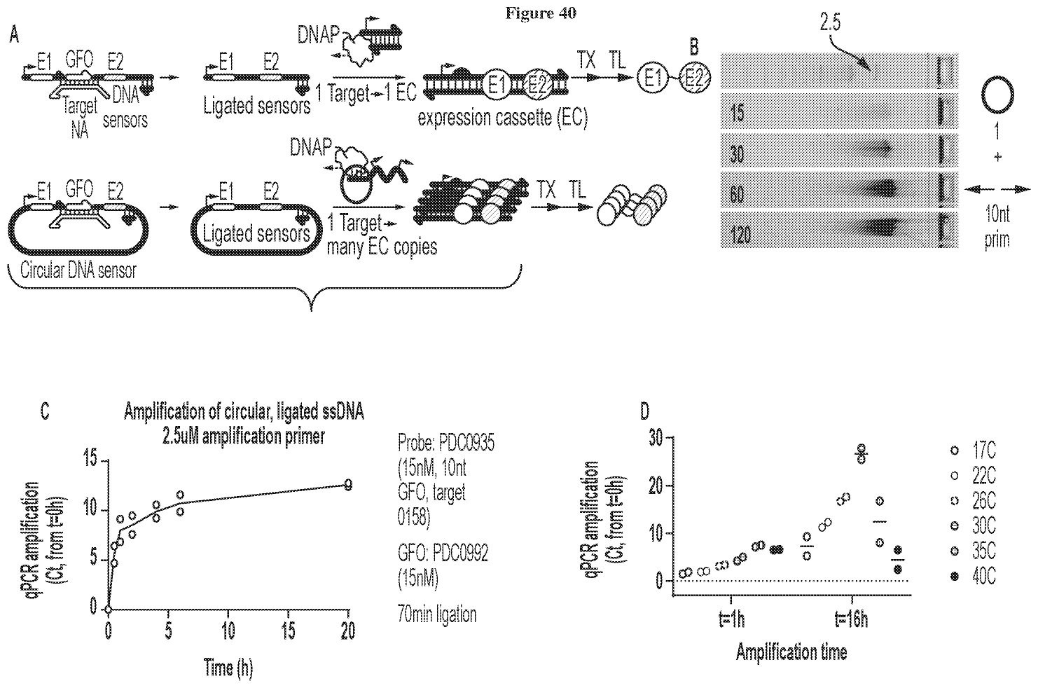

The present example further demonstrates first and second nucleic acid sensor parts in a circular configuration (e.g., FIG. 38; FIG. 40A) can enable improved sensitivity of technologies described herein. Without wishing to be bound by any one theory, a circular configuration can enable improved sensitivity via multiple-turnover amplification by a polymerase (e.g., DNA polymerase). A circular, single-stranded DNA fragment corresponding to a ligated first and second nucleic acid sensor parts (e.g., “probe”) targeting SARS-COV-2 RNA was generated and purified using CircLigase ssDNA-ssDNA ligase. The circular probe was then amplified by Phi29 DNA polymerase at 22° C., using a pair of 10 nucleotide primers (PDC1000 and PDC1007) to serve as initiation points for amplification. At defined time points, reaction aliquots were taken and heat-inactivated at 80° C. for 10 minutes, after which all time points were run on 2% agarose gels and imaged using SybrGold stain. In as little as 15 minutes, a high-molecular weight smear appeared, corresponding to amplified concatamers of the amplicon (FIG. 40B).

Amplified target nucleic acid was measured over time using quantitative Polymerase Chain Reaction (qPCR). Circular probes utilizing gap-filling oligos were used (see FIG. 43C). Probe(s) and GFOs were ligated by SplintR ligase in the presence of SARS-CoV-2 genomic RNA for 70 minutes, either at 22° C. (FIG. 40C) or a range of temperatures from 17° C. to 40° (FIG. 40D). At the indicated time points, reaction aliquots were taken and heat-inactivated at 80° C. for 10 minutes, after which all time points were analyzed to determine the amount of amplicon generated. Amplification increases over the course of a 20 hour reaction (FIG. 40C). Further qPCR-based assessment showed that increased temperature increases amplification rate, consistent with the optimal temperature (30° C.) for the DNA polymerase utilized (Phi29 DNA polymerase) (FIG. 40D).

Sequences utilized in FIG. 40 are summarized in Table 8.1.

TABLE 8.1
Exemplary sequences.

| Name | Sequence (5′ to 3′) | Description |
|---|---|---|
| PDC0954 | TAATAAGTAAGGAGGTTTTTTATGGATTACAAGGATGAT | Circular single- |
| | GATGATAAAACGACTCTGTCAGAGAGATTTTTAAGTGTG | stranded DNA |
| | TCACTTGTAATAAGTAATAGTGCGTGGTCACATCCTCAA | (used in FIG. 40B) |
| | TTCGAGAAGTAATAATACGACTCACTATAGGGAGGGAAG | |
| | TTTA | |
| PDC0935 | /5Phos/AAGTGTGTCACTTAGTGCGTGGTCACATCCTCA | Probe targeting |
| | ATTCGAGAAGGGCGGCGGGAGTGGAGGTGGCTCCGGTGG | SARS-CoV-2 RNA |
| | ATCTGCTTGGTCACACCCCCAATTCGAAAAATAATAACT | (used in FIG. 40C, D) |
| | AATACGACTCACTATAGGGAGGGAAGTTTATAATAAGTA | |
| | AGGAGGTTTTTTATGGATTACAAAGACCACGATGGTGAC | |
| | TACAAAGACCATGATATAGATTACAAGGATGATGATGAT | |
| | AAAACGACTCTGTCAG | |
| PDC0992 | agagaTTTTT | Gap-filling oligo |
| | | targeting SARS- |
| | | CoV-2 RNA (used |
| | | with PDC0935) |
| PDC1000 | CATCCTCA*A*T | Amplification |
| PDC1007 | TGACCACG*C*A | primers used in |
| | | FIG. 40 |
| | | (*Phosphorothioate |
| | | modification) |

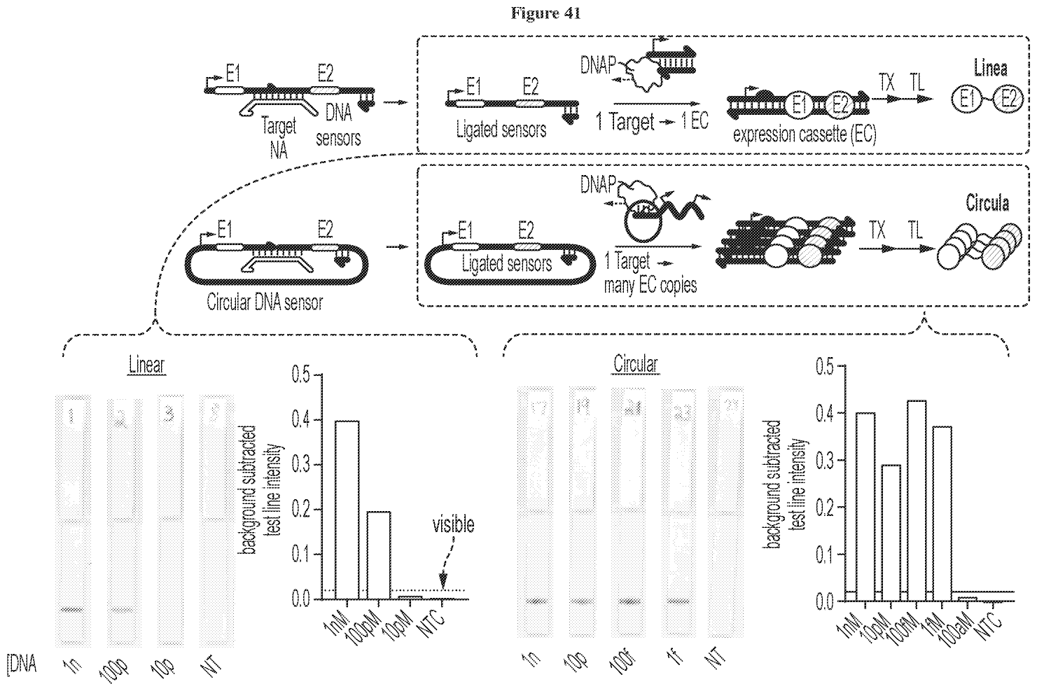

Exemplary linked (e.g., ligated) probes were generated and purified, corresponding to either a circular or non-circular probe configuration. Varying starting concentrations of these ligated products were then amplified with Phi29 at 22° C. for 2 hours. The resulting material was added to a cell-free protein synthesis reaction to generate a dual-epitope peptide (E1-E2), which in turn was detected by lateral flow half-strips. A circular configuration of first and second nucleic acid sensor parts can also increase sensitivity with a lateral-flow based read out. Circular probes show about 10⁵-fold improved sensitivity compared to a linear configuration when detected via an expressed peptide reporter (FIG. 41)

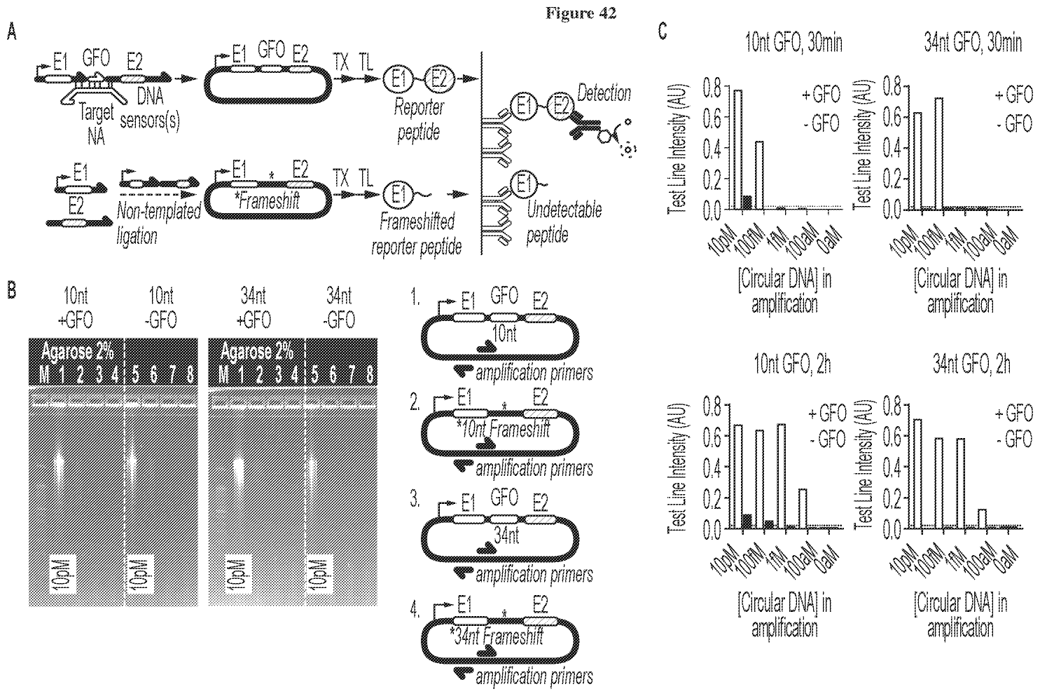

Exemplary experimentation demonstrated that gap-filling oligos (GFOs) can introduce a frameshift to provide, for example, proofreading. The degree of proofreading from a frameshifting gap-filling oligo was quantified. Four circular single-stranded DNA fragments, corresponding to ligated probes targeting SARS-COV-2 RNA, were generated and purified using CircLigase ssDNA-ssDNA ligase. Specifically, two pairs of circles corresponding to correctly-ligated (e.g., gap-filling oligo sequence correctly incorporated into the circle) and incorrectly-ligated (e.g., gap-filling oligo sequence not incorporated into the ligated circle) products were generated. Each of these circles were then amplified by Phi29 DNA polymerase at 22° C., using a pair of 10 nt primers (PDC1000 and PDC1007) to serve as initiation points for amplification. Importantly, these primers amplify all four circles with equivalent efficiency (FIG. 42B). These amplified circles were then added to a cell free protein synthesis reaction and detected via a lateral flow readout. As all circles were amplified with similar efficiency, any difference in detection (between +GFO and −GFO conditions) can be attributed to the effect of the frameshift introduced into the coding region when the GFO sequence is not incorporated. In each of the two examples, the 100− to >1000-fold difference in detection between +and −GFO circles of decreasing starting concentration demonstrating that this proofreading efficiently prevents detection of mis-linked products (FIG. 42C). Sequences utilized in FIG. 42 are summarized in Table 8.2.

TABLE 8.2
Exemplary sequences.

| Name | Sequence (5′ to 3′) | Description |
|---|---|---|
| PDC1000 | CATCCTCA*A*T | Amplification |
| PDC1007 | TGACCACG*C*A | primers used in |
| | | FIG. 40 |
| | | (*Phosphorothioate |
| | | modification) |

It was also determined that moving different sequence elements (e.g., promoters, ribosome binding sites, coding regions, and/or primer binding sites, etc.) onto GFOs can reduce signal from mis-ligation events. When a frameshift-inducing GFO is used in combination with a highly sensitive (e.g., typically in the sub-100 pM range (concentration of target nucleic acid in sample)) circularized first and second nucleic acid sensor parts, some background level signal (e.g., signal similar to that of a no target nucleic acid comprising sample). Circular probes utilizing gap-filling oligos of differing architectures were used (specifically, as shown in FIG. 43A-F). The individual sequence elements were the same across each probe, only differing in the distribution of elements across the two ends of a circular probe and the gap-filling oligo. In each case, probe and gap-filling oligo were ligated by SplintR ligase in the presence of SARS-COV-2 genomic RNA. Polymerization by Phi29 DNA polymerase was then carried out to form double-stranded DNA product, followed by expression in a cell-free extract, followed by peptide detection using lateral flow half-strips. The entire process was carried out at 22° C. FIG. 43G-L corresponds to the performance of each of the probe configurations shown in A-F. respectively, with initial concentration of SARS-COV-2 genomic RNA shown along the x-axis of each plot (NTC=no target nucleic acid control). Moving one or more sequence element off of the circular probe backbone and onto the gap-filling oligo (FIG. 43 H,I,J,L) reduces the intensity of the undesired background signal.

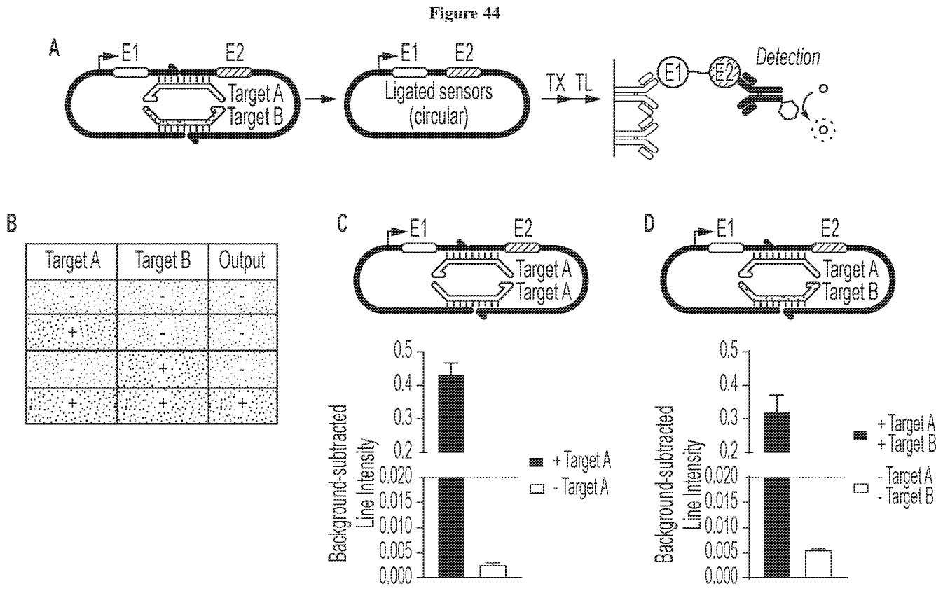

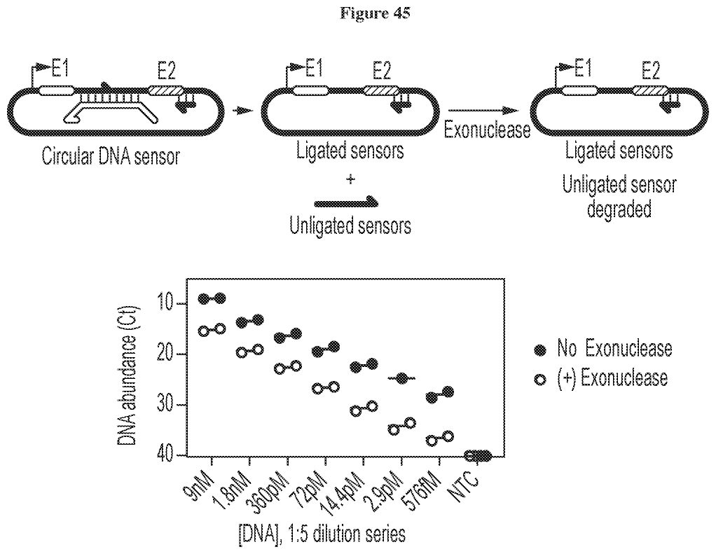

A plurality of regions that can hybridize to a target nucleic acid can be incorporated into first and second nucleic acid sensor parts. This example shows, among other things, one possible variation of the gap-filling oligo design approach. In this case, two distinct target RNA domains (A and B) are used to form a ligated circle, rather than a single continuous domain bridging a probe backbone with a GFO (FIG. 44A). This configuration promotes logic integration (e.g., wherein both of a target nucleic acid A and B must be present for a linked-product of the circular first and second nucleic acid sensor parts to be generated) (FIG. 44B). Probes comprising two halves of a circular reporter were ligated by SplintR ligase in the presence of SARS-COV-2 genomic RNA. Polymerization by Phi29 DNA polymerase was then carried out to form double-stranded DNA product, followed by expression in a cell-free extract, followed by peptide detection using lateral flow half-strips. The entire process was carried out at 22° C. This design generates a functional reporter architecture that can be configured to bind two copies of a single target (FIG. 44C) or two unique target domains (FIG. 44D). In this example, targets A and B are two subdomains of the SARS-COV-2 genome. Exonucleases can also be utilized to degrade non-linked (e.g., un-ligated) first and second nucleic acid sensor parts. A linear, single-stranded DNA fragment of varying concentration was incubated in the presence or absence of Exonuclease I for 1 hour at 22° C. The samples were then heat-inactivated at 80° C. for 10 minutes. A qPCR assay was then carried out to amplify and detect the presence of the full-length starting material: the difference in threshold cycle (Ct) between +Exo and −Exo conditions serves as an indication that the exonuclease successfully degraded the starting material. When linked-circular first and second nucleic acid sensor parts are hybridized to a target nucleic acid, excess non-linked circular first and second nucleic acid sensor parts remain that can be degraded by exonuclease enzymes (e.g., exonuclease I, exonuclease T, exonuclease VII, RecJf, exonuclease III, T7 exonuclease, exonuclease VII, lambda exonuclease, T5 exonuclease), while the linked-circular first and second nucleic acid sensor parts remain unaffected (FIG. 45).

### Example 9: Exemplary Structured Primers

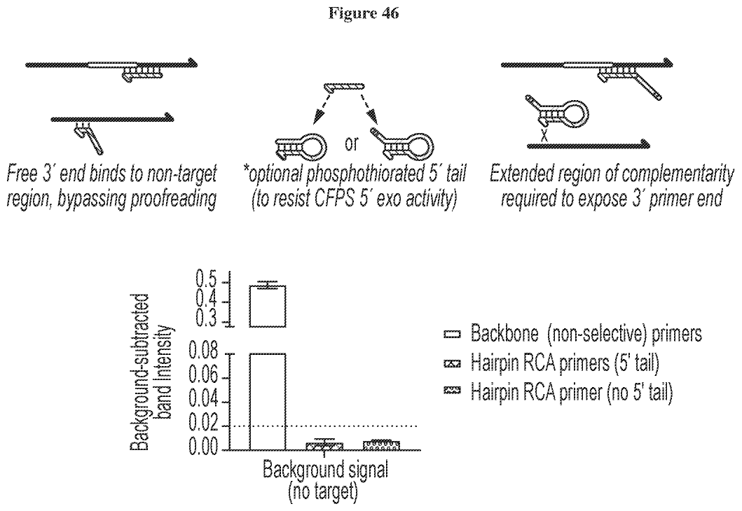

The present example demonstrates how amplification primers can be employed to reduce experimental background signal resulting from mis-priming events during polymerization. At reduced temperatures (about 16° C.-37° C.), and without wishing to be bound by any one theory, free 3′ ends can bind to a non-target region, bypassing any proofreading encoding in the primer sequence design. Added structure to the primer results in a cis-inhibited primer structure, which much interact via a longer region of sequence homology to expose the 3′ end and initiate polymerization. Probe and gap-filling oligo were incubated with SplintR ligase without any target RNA present. Polymerization by Phi29 DNA polymerase was then carried out to form double-stranded DNA product using either structured or unstructured primers, followed by expression in a cell-free extract, followed by peptide detection using lateral flow half-strips. The entire process was carried out at 22° C. Under these conditions, non-selective primers result in visible process background, but structured primers reduce this signal to a non-detectable level. To reduce off-target hybridization, the 3′ end of amplification primers can be blocked by an engineered cis-binding event, requiring a larger region of sequence complementarity to initiation a polymerization event (FIG. 46).

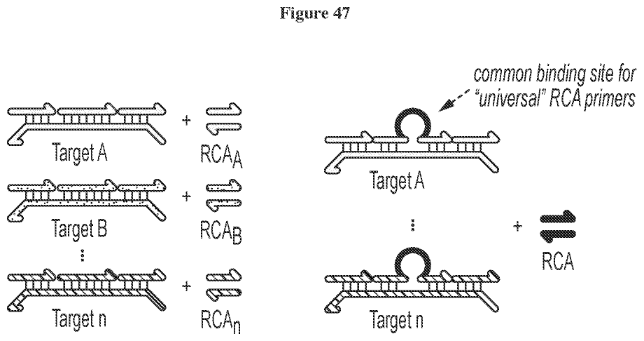

Amplification primers comprising sequences that can hybridize to a plurality of target nucleic acids (e.g., common amplification primers), can enable re-use of such amplification primers. Multiplexed detection reactions can comprises a plurality of primers, sequences that can hybridize to a plurality of target nucleic acids, enabling a single set of amplification proteins (e.g., for rolling circle amplification, PCR, etc.) to be used instead of a unique primer sequence for each target nucleic acid (FIG. 47).

### Example 10: Exemplary Detection

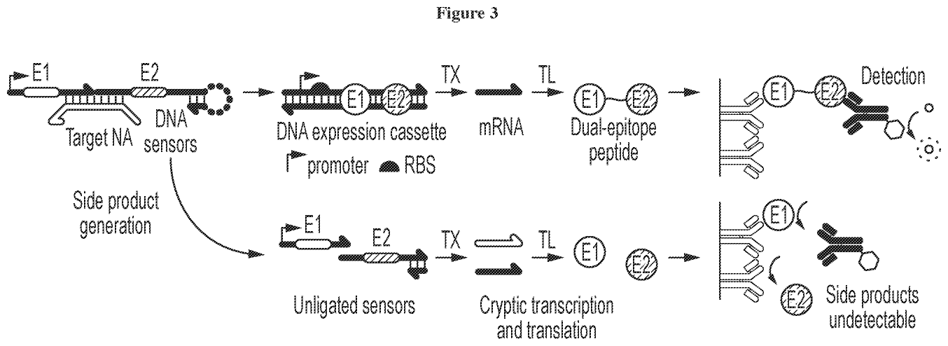

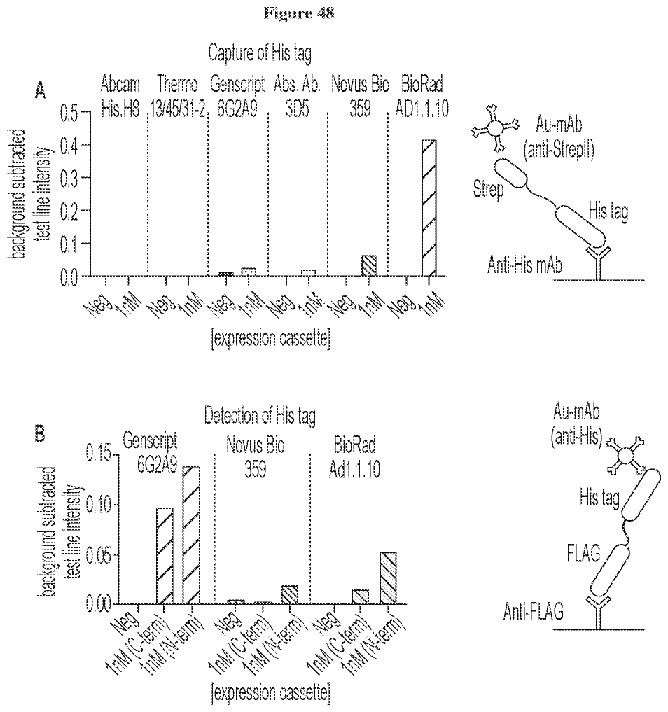

The present example demonstrates, among other things, validation of a His tag is a compatible element of a reporting element component (e.g., E1 and/or E2, FIG. 3), either as the capture (FIG. 48A) or detection (FIG. 48B) antibody of a sandwich-based lateral flow (LF) readout. Anti-His-tag monoclonal antibodies were functionalized on nitrocellulose membrane capture and peptide was expressed from 1 nM of a dsDNA expression cassette template with a His tag (FIG. 44A). Anti-His-tag monolocal antibodies were functionalized on colloidal gold yielded detection of peptide expressed from 1 nM of expression cassette template. When used as the detection antibody, the His tag can be placed on either the N-terminal or C-terminus of the peptide (FIG. 44B).

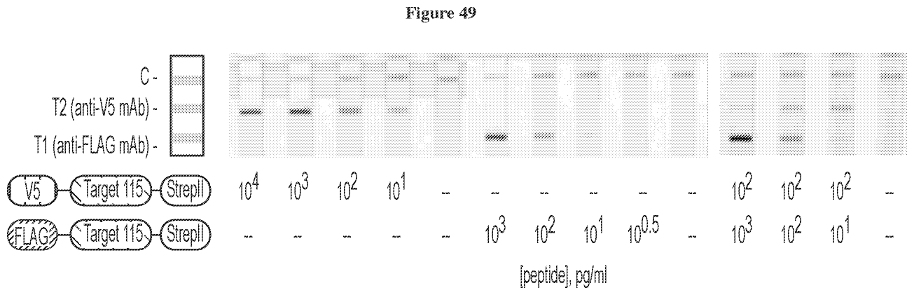

It was also determined that a lateral flow-based read out was highly specific for multiplexed dual-epitope peptide readouts. A lateral flow strip was developed to capture two representative dual-epitope peptide reporters, differing only in one of their two epitopes (V5-StrepII and FLAG-StrepII). Test lines were generated using anti-V5 and anti-FLAG antibodies, respectively, and a single StrepII detection antibody was employed for both peptides. Purified peptides were then detected by these strips, either individually or pooled. Appearance of test lines were found to be highly specific for their corresponding test lines (FIG. 49).

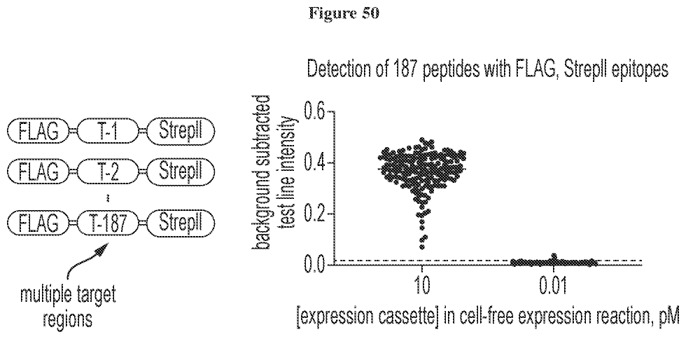

Dual-epitope peptide reporters were also found to be compatible with a wide array of target nucleic acid regions. dsDNA cassettes encoding FLAG-StrepII dual-epitope peptides with target regions against 187 unique subdomains of the SARS-COV-2 genome were expressed in a cell-free reaction and then detected by a lateral flow test strip. Every sensor variant was detectable at a concentration of 10 pM DNA in the cell-free reaction, demonstrating that this reporter configuration is robust against sequence changes (FIG. 50).

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A nucleic acid sensor system comprising at least a first nucleic acid sensor part and a second nucleic acid sensor part wherein:
(i) the first nucleic acid sensor part comprises:
(a) a sequence that is, encodes, or templates coding of, at least one first reporting element component; and
(b) a first target hybridization element; and
(ii) the second sensor part comprises:
(a) a sequence that is, encodes, or templates coding of, at least one second reporting element component; and
(b) a second target hybridization element, wherein:
the first and second target hybridization elements are related to one another in that, when the system is contacted with a sample comprising a target nucleic acid, hybridization of the target nucleic acid with both of the first and second target hybridization elements juxtaposes the first and second nucleic acid sensor parts with one another so that the juxtaposed parts are susceptible to linkage by one or more of:
(i) ligation to generate a ligation product;
(ii) templated copying to generate a linked template product.

2. The system of claim 1, wherein at least one of the first and second nucleic acid sensor parts includes a nucleic acid copying sequence element that directs synthesis of a reporter construct comprising the first and second reporting element components, or their complements.

3. The system of claim 2, wherein the nucleic acid copying sequence element is or comprises a promoter sequence element or its complement.

4. The system of claim 3, wherein transcription from the promoter sequence element or its complement generates the reporter construct.

5. The system of claim 2, wherein the nucleic acid copying sequence element is or comprises a primer binding site or its complement.

6. The system of claim 5, wherein extension of the primer generates the reporter construct.

7. The system of claim 2, wherein:
one of the first and second reporting element components binds to a capture sequence element; and
the other of the first and second reporting element components binds to a nucleic acid reporter.

8. The system of claim 4, wherein:
the first reporting element component encodes or templates coding of a first polypeptide element; and
the second reporting element component encodes or templates coding of a second polypeptide element,
so that translation of the reporter construct generates a polypeptide comprising the first and second polypeptide elements.

9. The system of claim 8, wherein at least one of the first and second polypeptide elements is or comprises an epitope

10. The system of claim 8, wherein at least one of the first and second polypeptide elements is or comprises a plurality of epitopes.

11. The system of claim 8, wherein the first and second polypeptide elements is each a complementing domain of a functional protein complex.

12-29. (canceled)

30. A nucleic acid sensor system comprising a plurality of a first nucleic acid sensor parts and a plurality of a second nucleic acid sensor parts wherein:
(i) each of the plurality of the first nucleic acid sensor parts comprises:
(a) a sequence that is, encodes, or templates coding of, at least one reporting element component; and
(b) a target hybridization element; and
(ii) each of the plurality of the second nucleic acid sensor parts comprises:
(a) a sequence that is, encodes, or templates coding of, at least one reporting element component; and
(b) a target hybridization element.

31-32. (canceled)

33. A method of detecting a target nucleic acid comprising the steps of:
(i) providing a sample comprising the target nucleic acid;
(ii) contacting the sample comprising the target nucleic acid with the nucleic acid sensor system of any of the preceding claims;
(iii) contacting the reaction product of step (ii) with conditions for linking the juxtaposed nucleic acid sensor parts under conditions favorable to the hybridization of the target nucleic acid to the target hybridization elements of the sensor system;
(iv) contacting the reaction product produced in step (iii) with a cell-free expression system, a strand displacing DNA Polymerase, and a primer, under conditions favorable to the production of a reporter.

34. The method of claim 33, further comprising detecting the presence/expression of the reporter of step (iv).

35. A kit comprising:
(i) a composition comprising a nucleic acid sensor system of claim 1, in a packaging material;
(ii) a sample collection device;
(iii) a positive control; and
(iv) instructions for use.

36. The nucleic acid sensor system of claim 1, wherein the first and second nucleic acid sensor parts are linked to form a circular probe.

37. The nucleic acid sensor system of claim 36, wherein the first and second nucleic acid sensor parts are directly linked.

38. The nucleic acid sensor system of claim 36, wherein the first and second nucleic acid sensor parts are indirectly linked.

39. The nucleic acid sensor system of claim 38, wherein the first and second nucleic acid sensor parts are linked by a third and fourth target hybridization element.

40. The nucleic acid sensor system of claim 38, wherein the first and second nucleic acid sensor parts are linked by a GFO.

41-46. (canceled)

47. The system of claim 1, wherein the system further comprises a third nucleic acid sensor part and a fourth nucleic acid sensor part, wherein
(iii) the third nucleic acid sensor part comprises:
(a) a sequence that is, encodes, or templates coding of, at least one third reporting element component; and
(b) a third target hybridization element; and
(iv) the fourth nucleic acid sensor part comprises:
(a) a sequence that is, encodes, or templates coding of, at least one fourth reporting element component; and
(b) a fourth target hybridization element, wherein:
the third and fourth target hybridization elements are related to one another in that, when the system is contacted with a sample comprising a target nucleic acid, hybridization of the target nucleic acid with both of the third and fourth target hybridization elements juxtaposes the third and fourth nucleic acid sensor parts with one another so that the juxtaposed parts are susceptible to linkage by one or more of:
(i) ligation to generate a ligation product;
(ii) templated copying to generate a linked template product, to generate a nucleic acid reporter.

48. The system of claim 47, wherein one of the first and second reporting element components includes a promoter sequence or complement thereof, and/or one of the third and fourth reporting element components includes a promoter sequence or complement thereof.

49. The system of claim 47, wherein the second nucleic acid reporter part is a protease.

50. The system of claim 47, wherein the system further comprises a fifth nucleic acid sensor part and a sixth nucleic acid sensor part, wherein
(v) the fifth nucleic acid sensor part comprises:
(a) a sequence that is, encodes, or templates coding of, at least one fifth reporting element component; and
(b) a fifth target hybridization element; and
(vi) the sixth nucleic acid sensor part comprises:
(a) a sequence that is, encodes, or templates coding of, at least one sixth reporting element component; and
(b) a sixth target hybridization element, wherein:
the fifth and sixth target hybridization elements are related to one another in that, when the system is contacted with a sample comprising a target nucleic acid, hybridization of the target nucleic acid with both of the fifth and sixth target hybridization elements juxtaposes the fifth and sixth nucleic acid sensor parts with one another so that the juxtaposed parts are susceptible to linkage by one or more of:
(i) ligation to generate a ligation product;
(ii) templated copying to generate a linked template product, to generate a nucleic acid reporter.


SEQUENCE LISTING (45 sequences)

SEQ ID NO: 1 - DNA, 160 bp, Artificial Sequence
  source: /note=“Description of Artificial Sequence Synthetic polynucleotide”
taataagtaaggaggttttttatggattacaaggatgatgatgataaaacgactctgtcagagagatttttaagtgtgtcacttgtaataagtaatagtgcgtggtcacatcctcaattcgagaagtaataatacgactcactatagggagggaagttta

SEQ ID NO: 2 - DNA, 244 bp, Artificial Sequence
  source: /note=“Description of Artificial Sequence Synthetic polynucleotide”
aagtgtgtcacttagtgcgtggtcacatcctcaattcgagaagggcggcgggagtggaggtggctccggtggatctgcttggtcacacccccaattcgaaaaataataactaatacgactcactatagggagggaagtttataataagtaaggaggttttttatggattacaaagaccacgatggtgactacaaagaccatgatatagattacaaggatgatgatgataaaacgactctgtcag

SEQ ID NO: 3 - DNA, 10 bp, Artificial Sequence
  source: /note=“Description of Artificial Sequence Synthetic oligonucleotide”
agagattttt

SEQ ID NO: 4 - DNA, 10 bp, Artificial Sequence
  source: /note=“Description of Artificial Sequence Synthetic oligonucleotide”
catcctcaat

SEQ ID NO: 5 - DNA, 10 bp, Artificial Sequence
  source: /note=“Description of Artificial Sequence Synthetic oligonucleotide”
tgaccacgca

SEQ ID NO: 6 - DNA, 190 bp, Artificial Sequence
  source: /note=“Description of Artificial Sequence Synthetic polynucleotide”
  modified_base (83)..(83): a, c, t, g, unknown or other
  modified_base (85)..(87): a, c, t, g, unknown or other
  modified_base (90)..(92): a, c, t, g, unknown or other
  modified_base (94)..(96): a, c, t, g, unknown or other
  modified_base (99)..(101): a, c, t, g, unknown or other
  modified_base (103)..(105): a, c, t, g, unknown or other
  modified_base (108)..(110): a, c, t, g, unknown or other
  modified_base (112)..(114): a, c, t, g, unknown or other
  modified_base (117)..(118): a, c, t, g, unknown or other
ggacggaagtttataataagtaaggaggttttttatgggattgaacgacatcttcgaggcccagaaaattgagtggcatgagnynnnyvnnnynnnyvnnnynnnyvnnnynnnyvnngattataaagaccacgacggtgattataaggaccacgacattgattacaaagatgatgatgataaataataa

SEQ ID NO: 7 - DNA, 59 bp, Artificial Sequence
  source: /note=“Description of Artificial Sequence Synthetic oligonucleotide”
gaattaatacgactcactatagggacggaagtttataataagtaaggaggttttttatg

SEQ ID NO: 8 - DNA, 40 bp, Artificial Sequence
  source: /note=“Description of Artificial Sequence Synthetic oligonucleotide”
ttattatttatcatcatcatctttgtaatcaatgtcgtgg

SEQ ID NO: 9 - DNA, 200 bp, Artificial Sequence
  source: /note=“Description of Artificial Sequence Synthetic polynucleotide”
  modified_base (83)..(83): a, c, t, g, unknown or other
  modified_base (85)..(87): a, c, t, g, unknown or other
  modified_base (90)..(92): a, c, t, g, unknown or other
  modified_base (94)..(96): a, c, t, g, unknown or other
  modified_base (99)..(101): a, c, t, g, unknown or other
  modified_base (103)..(105): a, c, t, g, unknown or other
  modified_base (108)..(110): a, c, t, g, unknown or other
  modified_base (112)..(114): a, c, t, g, unknown or other
  modified_base (117)..(118): a, c, t, g, unknown or other
ggacggaagtttataataagtaaggaggttttttatgggattgaacgacatcttcgaggcccagaaaattgagtggcatgagnynnnyvnnnynnnyvnnnynnnyvnnnynnnyvnntacccctacgatgtccccgactacgctgggtatccgtatgatgtaccagattacgccgggtacccttacgatgtgcctgact

SEQ ID NO: 10 - DNA, 34 bp, Artificial Sequence
  source: /note=“Description of Artificial Sequence Synthetic oligonucleotide”
ttattacgcatagtcaggcacatcgtaagggtac

SEQ ID NO: 11 - DNA, 200 bp, Artificial Sequence
  source: /note=“Description of Artificial Sequence Synthetic polynucleotide”
  modified_base (83)..(83): a, c, t, g, unknown or other
  modified_base (85)..(87): a, c, t, g, unknown or other
  modified_base (90)..(92): a, c, t, g, unknown or other
  modified_base (94)..(96): a, c, t, g, unknown or other
  modified_base (99)..(101): a, c, t, g, unknown or other
  modified_base (103)..(105): a, c, t, g, unknown or other
  modified_base (108)..(110): a, c, t, g, unknown or other
  modified_base (112)..(114): a, c, t, g, unknown or other
  modified_base (117)..(118): a, c, t, g, unknown or other
ggacggaagtttataataagtaaggaggttttttatgggattgaacgacatcttcgaggcccagaaaattgagtggcatgagnynnnyvnnnynnnyvnnnynnnyvnnnynnnyvnngagcaaaagttaatctctgaagaagacctggaacagaaacttatctctgaggaagatttagagcaaaaactgatctcagaag

SEQ ID NO: 12 - DNA, 46 bp, Artificial Sequence
  source: /note=“Description of Artificial Sequence Synthetic oligonucleotide”
ttattaaaggtcttcttctgagatcagtttttgctctaaatcttcc

SEQ ID NO: 13 - DNA, 200 bp, Artificial Sequence
  source: /note=“Description of Artificial Sequence Synthetic polynucleotide”
  modified_base (83)..(83): a, c, t, g, unknown or other
  modified_base (85)..(87): a, c, t, g, unknown or other
  modified_base (90)..(92): a, c, t, g, unknown or other
  modified_base (94)..(96): a, c, t, g, unknown or other
  modified_base (99)..(101): a, c, t, g, unknown or other
  modified_base (103)..(105): a, c, t, g, unknown or other
  modified_base (108)..(110): a, c, t, g, unknown or other
  modified_base (112)..(114): a, c, t, g, unknown or other
  modified_base (117)..(118): a, c, t, g, unknown or other
ggacggaagtttataataagtaaggaggttttttatgggattgaacgacatcttcgaggcccagaaaattgagtggcatgagnynnnyvnnnynnnyvnnnynnnyvnnnynnnyvnnggcaaacccatccccaatccgctgttagggctggatagcacaggaggtaagcccattcctaacccactgttgggcttggata

SEQ ID NO: 14 - DNA, 77 bp, Artificial Sequence
  source: /note=“Description of Artificial Sequence Synthetic oligonucleotide”
ttattaggttgaatccaaacccagtaacgggttcgggataggtttaccaccagtactatccaagcccaacagtgggt

SEQ ID NO: 15 - DNA, 148 bp, Artificial Sequence
  source: /note=“Description of Artificial Sequence Synthetic polynucleotide”
  modified_base (31)..(32): a, c, t, g, unknown or other
  modified_base (35)..(37): a, c, t, g, unknown or other
  modified_base (39)..(41): a, c, t, g, unknown or other
  modified_base (44)..(46): a, c, t, g, unknown or other
  modified_base (48)..(50): a, c, t, g, unknown or other
  modified_base (53)..(55): a, c, t, g, unknown or other
  modified_base (57)..(59): a, c, t, g, unknown or other
  modified_base (62)..(64): a, c, t, g, unknown or other
  modified_base (66)..(66): a, c, t, g, unknown or other
ttattacttatcgtcgtcatccttgtaatcnnvynnnynnnvynnnynnnvynnnynnnvynnnynctcatgccactcaattttctgggcctcgaagatgtcgttcaatcccataaaaaacctccttacttattataaacttccgtcc

SEQ ID NO: 16 - DNA, 151 bp, Artificial Sequence
  source: /note=“Description of Artificial Sequence Synthetic polynucleotide”
  modified_base (34)..(35): a, c, t, g, unknown or other
  modified_base (38)..(40): a, c, t, g, unknown or other
  modified_base (42)..(44): a, c, t, g, unknown or other
  modified_base (47)..(49): a, c, t, g, unknown or other
  modified_base (51)..(53): a, c, t, g, unknown or other
  modified_base (56)..(58): a, c, t, g, unknown or other
  modified_base (60)..(62): a, c, t, g, unknown or other
  modified_base (65)..(67): a, c, t, g, unknown or other
  modified_base (69)..(69): a, c, t, g, unknown or other
ttattacgcataatccggaacgtcgtagggatannvynnnynnnvynnnynnnvynnnynnnvynnnynctcatgccactcaattttctgggcctcgaagatgtcgttcaatcccataaaaaacctccttacttattataaacttccgtcc

SEQ ID NO: 17 - DNA, 154 bp, Artificial Sequence
  source: /note=“Description of Artificial Sequence Synthetic polynucleotide”
  modified_base (37)..(38): a, c, t, g, unknown or other
  modified_base (41)..(43): a, c, t, g, unknown or other
  modified_base (45)..(47): a, c, t, g, unknown or other
  modified_base (50)..(52): a, c, t, g, unknown or other
  modified_base (54)..(56): a, c, t, g, unknown or other
  modified_base (59)..(61): a, c, t, g, unknown or other
  modified_base (63)..(65): a, c, t, g, unknown or other
  modified_base (68)..(70): a, c, t, g, unknown or other
  modified_base (72)..(72): a, c, t, g, unknown or other
ttattaaaggtcctcctccgaaatcaatttctgttcnnvynnnynnnvynnnynnnvynnnynnnvynnnynctcatgccactcaattttctgggcctcgaagatgtcgttcaatcccataaaaaacctccttacttattataaacttccgtcc

SEQ ID NO: 18 - DNA, 166 bp, Artificial Sequence
  source: /note=“Description of Artificial Sequence Synthetic polynucleotide”
  modified_base (49)..(50): a, c, t, g, unknown or other
  modified_base (53)..(55): a, c, t, g, unknown or other
  modified_base (57)..(59): a, c, t, g, unknown or other
  modified_base (62)..(64): a, c, t, g, unknown or other
  modified_base (66)..(68): a, c, t, g, unknown or other
  modified_base (71)..(73): a, c, t, g, unknown or other
  modified_base (75)..(77): a, c, t, g, unknown or other
  modified_base (80)..(82): a, c, t, g, unknown or other
  modified_base (84)..(84): a, c, t, g, unknown or other
ttattatgtcgaatccagaccaagaagcgggtttggaattggtttcccnnvynnnynnnvynnnynnnvynnnynnnvynnnynctcatgccactcaattttctgggcctcgaagatgtcgttcaatcccataaaaaacctccttacttattataaacttccgtcc

SEQ ID NO: 19 - DNA, 257 bp, Artificial Sequence
  source: /note=“Description of Artificial Sequence Synthetic polynucleotide”
gaattaatacgactcactatagggacggaagtttataataagtaaggaggttttttatggattacaaagaccacgatggtgactacaaagaccatgatatagattacaaggatgatgatgataaaaattatctatgcattactaacccttccgccactaaaagtgcgtggtcacatcctcaattcgagaagggcggcgggagtggaggtggctccggtggatctgcttggtcacacccccaattcgaaaaataataa

SEQ ID NO: 20 - DNA, 143 bp, Artificial Sequence
  source: /note=“Description of Artificial Sequence Synthetic polynucleotide”
gaattaatacgactcactatagggacggaagtttataataagtaaggaggttttttatggattacaaagaccacgatggtgactacaaagaccatgatatagattacaaggatgatgatgataaaaattatctatgcattact

SEQ ID NO: 21 - DNA, 114 bp, Artificial Sequence
  source: /note=“Description of Artificial Sequence Synthetic polynucleotide”
aacccttccgccactaaaagtgcgtggtcacatcctcaattcgagaagggcggcgggagtggaggtggctccggtggatctgcttggtcacacccccaattcgaaaaataataa

SEQ ID NO: 22 - DNA, 23 bp, Artificial Sequence
  source: /note=“Description of Artificial Sequence Synthetic oligonucleotide”
ttattatttttcgaattgggggt

SEQ ID NO: 23 - DNA, 64 bp, Chlamydia trachomatis
tgtttcggcaattgtttagtggcggaagggttagtaatgcatagataatttgtccttaacttgg

SEQ ID NO: 24 - DNA, 626 bp, Artificial Sequence
  source: /note=“Description of Artificial Sequence Synthetic polynucleotide”
gaattaatacgactcactatagggacggaagtttataataagtaaggaggttttttatggactacaaagacgatgacgacaaggtatttactctacgcgacttcgtaggcgattggcgccagaccgcaggctacaacttagaccaagtcctagaacagggcggtgtttcaagcttattccaaaacctaggagtaagcgtaacgccgatccaacgcattgttctatcgggcgaaaatggcctcaaaatcgatattcatgtgatcattccatacgaggggttatccggagaccagatgggacagatcgaaaagatcttcaaagtagtctaccccgtggacgatcaccatttcaaggttatccttcattatggaacgcttgtcatcgacggggtcaccccgaacatgattgactactttggtcgtccttatgaaggaattgcggtctttgacgggaagaagattactgttacagggaccctttggaacggtaataaaatcattgacgagcgtctgatcaaccccgatggaagtcttctttttcgtgtgactatcaacggagtgactggttggcgcctgtgtgaacgcattcttgctaactggagtcacccccaatttgaaaaataataa

SEQ ID NO: 25 - DNA, 599 bp, Artificial Sequence
  source: /note=“Description of Artificial Sequence Synthetic polynucleotide”
gaattaatacgactcactatagggacggaagtttataataagtaaggaggttttttatggactacaaagacgatgacgacaaggtatttactctacgcgacttcgtaggcgattggcgccagaccgcaggctacaacttagaccaagtcctagaacagggcggtgtttcaagcttattccaaaacctaggagtaagcgtaacgccgatccaacgcattgttctatcgggcgaaaatggcctcaaaatcgatattcatgtgatcattccatacgaggggttatccggagaccagatgggacagatcgaaaagatcttcaaagtagtctaccccgtggacgatcaccatttcaaggttatccttcattatggaacgcttgtcatcgacggggtcaccccgaacatgattgactactttggtcgtccttatgaaggaattgcggtctttgacgggaagaagattactgttacagggaccctttggaacggtaataaaatcattgacgagcgtctgatcaaccccgatggaagtcttctttttcgtgtgactatcaacggagtgactggttggcgcctgtgtgaacgcattcttgcttaataa

SEQ ID NO: 26 - DNA, 602 bp, Artificial Sequence
  source: /note=“Description of Artificial Sequence Synthetic polynucleotide”
gaattaatacgactcactatagggacggaagtttataataagtaaggaggttttttatggtatttactctacgcgacttcgtaggcgattggcgccagaccgcaggctacaacttagaccaagtcctagaacagggcggtgtttcaagcttattccaaaacctaggagtaagcgtaacgccgatccaacgcattgttctatcgggcgaaaatggcctcaaaatcgatattcatgtgatcattccatacgaggggttatccggagaccagatgggacagatcgaaaagatcttcaaagtagtctaccccgtggacgatcaccatttcaaggttatccttcattatggaacgcttgtcatcgacggggtcaccccgaacatgattgactactttggtcgtccttatgaaggaattgcggtctttgacgggaagaagattactgttacagggaccctttggaacggtaataaaatcattgacgagcgtctgatcaaccccgatggaagtcttctttttcgtgtgactatcaacggagtgactggttggcgcctgtgtgaacgcattcttgctaactggagtcacccccaatttgaaaaataataa

SEQ ID NO: 27 - DNA, 575 bp, Unknown
  source: /note=“Description of Unknown NanoLuc sequence”
gaattaatacgactcactatagggacggaagtttataataagtaaggaggttttttatggtatttactctacgcgacttcgtaggcgattggcgccagaccgcaggctacaacttagaccaagtcctagaacagggcggtgtttcaagcttattccaaaacctaggagtaagcgtaacgccgatccaacgcattgttctatcgggcgaaaatggcctcaaaatcgatattcatgtgatcattccatacgaggggttatccggagaccagatgggacagatcgaaaagatcttcaaagtagtctaccccgtggacgatcaccatttcaaggttatccttcattatggaacgcttgtcatcgacggggtcaccccgaacatgattgactactttggtcgtccttatgaaggaattgcggtctttgacgggaagaagattactgttacagggaccctttggaacggtaataaaatcattgacgagcgtctgatcaaccccgatggaagtcttctttttcgtgtgactatcaacggagtgactggttggcgcctgtgtgaacgcattcttgcttaataa

SEQ ID NO: 28 - DNA, 731 bp, Artificial Sequence
  source: /note=“Description of Artificial Sequence Synthetic polynucleotide”
gaattaatacgactcactatagggacggaagtttataataagtaaggaggttttttatggattacaaagaccacgatggtgactacaaagaccatgatatagattacaaggatgatgatgataaagtatttactctacgcgacttcgtaggcgattggcgccagaccgcaggctacaacttagaccaagtcctagaacagggcggtgtttcaagcttattccaaaacctaggagtaagcgtaacgccgatccaacgcattgttctatcgggcgaaaatggcctcaaaatcgatattcatgtgatcattccatacgaggggttatccggagaccagatgggacagatcgaaaagatcttcaaagtagtctaccccgtggacgatcaccatttcaaggttatccttcattatggaacgcttgtcatcgacggggtcaccccgaacatgattgactactttggtcgtccttatgaaggaattgcggtctttgacgggaagaagattactgttacagggaccctttggaacggtaataaaatcattgacgagcgtctgatcaaccccgatggaagtcttctttttcgtgtgactatcaacggagtgactggttggcgcctgtgtgaacgcattcttgctagtgcgtggtcacatcctcaattcgagaagggcggcgggagtggaggtggctccggtggatctgcttggtcacacccccaattcgaaaaataataa

SEQ ID NO: 29 - RNA, 64 nt, Severe acute respiratory syndrome-related coronavirus 2
cugcuucagacacuuaugccuguuggcaucauucuauuggauuugauuacgucuauaauccguu

SEQ ID NO: 30 - RNA, 64 nt, Severe acute respiratory syndrome-related coronavirus 2
ucguuccucaucacguagucgcaacaguucaagaaauucaacuccaggcagcaguaggggaacu

SEQ ID NO: 31 - RNA, 64 nt, Influenza A virus
cauccgaucacaauuggaaaauguccaaaauauguaaaaagcacaaaauugagacuggccacag

SEQ ID NO: 32 - DNA, 167 bp, Artificial Sequence
  source: /note=“Description of Artificial Sequence Synthetic polynucleotide”
gaattaatacgactcactatagggacggaagtttataataagtaaggaggttttttatggagcagaaacttatttcggaggaagacttggagcaaaaactgatcagtgaggaagacttagaacagaagttaatcagtgaggaagatctgcggattatagacgtaatc

SEQ ID NO: 33 - DNA, 114 bp, Artificial Sequence
  source: /note=“Description of Artificial Sequence Synthetic polynucleotide”
aaatccaatagaatgatgagtgcgtggtcacatcctcaattcgagaagggcggcgggagtggaggtggctccggtggatctgcttggtcacacccccaattcgaaaaataataa

SEQ ID NO: 34 - DNA, 167 bp, Artificial Sequence
  source: /note=“Description of Artificial Sequence Synthetic polynucleotide”
gaattaatacgactcactatagggacggaagtttataataagtaaggaggttttttatgggcaagcctatccccaatccgctgttgggactggatagcactggtgggaaacctatcccgaacccacttcttggcttggactccaccgggctggagttgaatttcttg

SEQ ID NO: 35 - DNA, 114 bp, Artificial Sequence
  source: /note=“Description of Artificial Sequence Synthetic polynucleotide”
aactgttgcgactacgtgagtgcgtggtcacatcctcaattcgagaagggcggcgggagtggaggtggctccggtggatctgcttggtcacacccccaattcgaaaaataataa

SEQ ID NO: 36 - DNA, 164 bp, Artificial Sequence
  source: /note=“Description of Artificial Sequence Synthetic polynucleotide”
gaattaatacgactcactatagggacggaagtttataataagtaaggaggttttttatgtacccctatgatgttcctgactatgctgggtatccttacgatgtccccgactacgccgggtatccgtatgacgtacctgattatgctaattttgtgctttttaca

SEQ ID NO: 37 - DNA, 114 bp, Artificial Sequence
  source: /note=“Description of Artificial Sequence Synthetic polynucleotide”
tattttggacattttccaagtgcgtggtcacatcctcaattcgagaagggcggcgggagtggaggtggctccggtggatctgcttggtcacacccccaattcgaaaaataataa

SEQ ID NO: 38 - DNA, 235 bp, Artificial Sequence
  source: /note=“Description of Artificial Sequence Synthetic polynucleotide”
taccagcctgtacgtaataagtaatagtgcgtggtcacatcctcaattcgagaagggcggcgggagtggaggtggctccggtggatctgcttggtcacacccccaattcgaaaaataataaagggaagtttataataagtaaggaggttttttatggattacaaagaccacgatggtgactacaaagaccatgatatagattacaaggatgatgatgataaaaagtacacaattt

SEQ ID NO: 39 - DNA, 235 bp, Artificial Sequence
  source: /note=“Description of Artificial Sequence Synthetic polynucleotide”
ggcaggcacttctgtaataagtaatagtgcgtggtcacatcctcaattcgagaagggcggcgggagtggaggtggctccggtggatctgcttggtcacacccccaattcgaaaaataataaagggaagtttataataagtaaggaggttttttatggattacaaagaccacgatggtgactacaaagaccatgatatagattacaaggatgatgatgataaattagcagcatcta

SEQ ID NO: 40 - DNA, 235 bp, Artificial Sequence
  source: /note=“Description of Artificial Sequence Synthetic polynucleotide”
aagatctgaatcggtaataagtaatagtgcgtggtcacatcctcaattcgagaagggcggcgggagtggaggtggctccggtggatctgcttggtcacacccccaattcgaaaaataataaagggaagtttataataagtaaggaggttttttatggattacaaagaccacgatggtgactacaaagaccatgatatagattacaaggatgatgatgataaagcacaatcaccaa

SEQ ID NO: 41 - DNA, 235 bp, Artificial Sequence
  source: /note=“Description of Artificial Sequence Synthetic polynucleotide”
cgatgagagattcgtaataagtaatagtgcgtggtcacatcctcaattcgagaagggcggcgggagtggaggtggctccggtggatctgcttggtcacacccccaattcgaaaaataataaagggaagtttataataagtaaggaggttttttatggattacaaagaccacgatggtgactacaaagaccatgatatagattacaaggatgatgatgataaaccatggccatttt

SEQ ID NO: 42 - DNA, 55 bp, Artificial Sequence
  source: /note=“Description of Artificial Sequence Synthetic oligonucleotide”
tgcatagaatgtccaatctaatacgactcactatagggaaccctgagttgaacat

SEQ ID NO: 43 - DNA, 55 bp, Artificial Sequence
  source: /note=“Description of Artificial Sequence Synthetic oligonucleotide”
cagcaaaagcacagaaactaatacgactcactataggggataatacagttgaatt

SEQ ID NO: 44 - DNA, 55 bp, Artificial Sequence
  source: /note=“Description of Artificial Sequence Synthetic oligonucleotide”
tcaaagttgaatctgcactaatacgactcactatagggtcagagacaaagtcatt

SEQ ID NO: 45 - DNA, 55 bp, Artificial Sequence
  source: /note=“Description of Artificial Sequence Synthetic oligonucleotide”
atatactgctcatacttctaatacgactcactatagggtccaagttcttggagat